SEQUENCE LISTING (93 sequences)

SEQ ID NO: 1 - protein, 1629 aa, Francisella sp.
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 2 - protein, 1629 aa, synthetic construct
  REGION 1..1629: note = Protein sequence of recombinant dFnCas9 from Francisella novicida
MNFKILPIAIALGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDAIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 3 - protein, 1899 aa, synthetic construct
  REGION 1..1899: note = Protein sequence of recombinant dFnCas9 from Francisella novicida coupled with Green Fluorescent Protein
VSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKGIEENLYFQSNAMYPYDVPDYASPKKKRKVEASNFKILPIAIALGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDAIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 4 - DNA, 502 bp, synthetic construct
  misc_feature 1..502: note = Synthetic DNA containing portion of human gene encoding for Glycogen storage disease type I
  source 1..502: mol_type = other DNA
agcataggagatggaatttgttaaataaatgaattatggataacgaatggatggtaagatgggtggatggatggggggtgaacggatggatggggggtgaatggatggatgaatgggtagatgggtggatagggggatggctgggtggctgggtagatgatgcactgtctcccagatgaggaccttttcacctttactccattctctttcctgccctttagggagcccctctggccatgccatgggcacaacaggtgtatactacgtgatggtcacatctactctttccatctttcagggaaagataaagccgacctacagatttcggtaagaactcaccactggggtgtaggtggtggagggcaggaggcagctctctctgtagctgacacaccacgtattcttcctcacatccccctagcccgctcccacacctgggcagccgctgattaagagttgtggcactttggatagggataaacctcagagtcagggaatgttt

SEQ ID NO: 5 - DNA, 20 bp, synthetic construct
  misc_feature 1..20: note = Synthetic DNA for SgRNA seq.1 against Glycogen storage disease type I
  source 1..20: mol_type = other DNA
ggccatgccatgggaacaac

SEQ ID NO: 6 - DNA, 20 bp, synthetic construct
  misc_feature 1..20: note = Synthetic DNA for SgRNA seq.2 against Glycogen storage disease type I
  source 1..20: mol_type = other DNA
ggccatgccatgagcacaac

SEQ ID NO: 7 - DNA, 502 bp, synthetic construct
  misc_feature 1..502: note = Synthetic DNA containing portion of human gene encoding for Glanzmann thrombosthenia
  source 1..502: mol_type = other DNA
tcccagggaccaagcgtggcccacagaggcccacagcacacccggacccctctccctcctcccatcccctctgccccgtgggtcccggggaggccgggccagagaccagagagcctgctcactacgagaactggatcctgaagcctcgagggctgctcgggctctggcaggaagatctgtctgcgatcccgcttgtgatgggccgggtgaatgggggaggggctggggatgggcagcccccagtccacctcggggggcaaaggagtggtcaggcccaggtctcccccgaaccccagcccacagaggtgccccggtggttggtctggggccgccttcccaggtctttcttccacccagctcttaccttgagagggttgacaggaggctgtgggaagcactgaaggcccccctggggctgtatatccaggatgtagagcaggtcggagggctgggactgtcccggaaggtggatgctgaggtgaagaccattcacagtcccagg

SEQ ID NO: 8 - DNA, 20 bp, synthetic construct
  misc_feature 1..20: note = Synthetic DNA for SgRNA seq.1 against Glanzmann thrombasthenia
  source 1..20: mol_type = other DNA
gcagcccccagtccgcctcg

SEQ ID NO: 9 - DNA, 20 bp, synthetic construct
  misc_feature 1..20: note = Synthetic DNA for SgRNA seq.2 against Glanzmann thrombasthenia
  source 1..20: mol_type = other DNA
gcagcccccagtacacctcg

SEQ ID NO: 10 - DNA, 500 bp, synthetic construct
  misc_feature 1..500: note = Synthetic DNA containing portion of human gene encoding for X-linked myotubular myopathy
  source 1..500: mol_type = other DNA
catggaagtatatattatttcctctctgagaaacttggcatttagaaggcacattgctgaattgtattgtcacattttgtgttatatgctttctcagttttgtacccattaattaaaacaaattatcttcatcaatttattcagcgaataggtcatggtgataaaaaccacaccgatgctgaccgttctcctatttttctccagtttattgattgtgtgtggcaaatgtcaaaacaggcaaggaatatgagggatgaaaatacattcaactcattgtttaaattaaacattttaatataatgattgggatttgaccccaaaataatacagggtagagctagagatgaagcaagactgggcatgtgtgaatacttgcagaagccaggtacatgagcgttcacctttttattcctgctactattgatatatctgggattttccctaataaagttttaaaaattaagtatttaactctgatgtctctactagtggaattgaca

SEQ ID NO: 11 - DNA, 20 bp, synthetic construct
  misc_feature 1..20: note = Synthetic DNA for SgRNA seq.1 against X-linked myotubular myopathy
  source 1..20: mol_type = other DNA
ggcaaatgtcaaaaaaggca

SEQ ID NO: 12 - DNA, 20 bp, synthetic construct
  misc_feature 1..20: note = Synthetic DNA for SgRNA seq.2 against X-linked myotubular myopathy
  source 1..20: mol_type = other DNA
ggcaaatgtcaagacaggca

SEQ ID NO: 13 - DNA, 502 bp, synthetic construct
  misc_feature 1..502: note = Synthetic DNA containing portion of human gene encoding for Hereditary Factor VIII deficiency disease(HA)
  source 1..502: mol_type = other DNA
tgcaggactgatgatagttaattcaggaggcttcaaggcagtgtctgctaggatttagcacaaaggtagaaggcaagccagggagggacactgccctggagctgaggagggagaggtgacggcagtggcaggtgctgcagtggccaccctcagtagaggtcctgtgcctcgcagcccagaacctccatcctcagggcaatctggtgcacccaactctgggggtgaattcgaaggtagcgagtcagtaacgatgggtctagagagttcaccacaggtgtgaaggagtcttgatttccctgaaaaacctgaaagaggaaagatagcatttattggttgtctgacaggacaatggtcactgcaaagcacagacgcttttctcacttctagttgtttccagtagtccaggattaaaccctggaagatgactagatgactctgtcctacgcatctgggacatctagaattaagagcaccctgctttggaggccttcactctagctag

SEQ ID NO: 14 - DNA, 20 bp, synthetic construct
  misc_feature 1..20: note = Synthetic DNA for SgRNA seq 1 against Hereditary factor VIII deficiency disease(HA)
  source 1..20: mol_type = other DNA
ggtagcgagtcagtgacgat

SEQ ID NO: 15 - DNA, 20 bp, synthetic construct
  misc_feature 1..20: note = Synthetic DNA for SgRNA seq.2 against Hereditary factor VIII deficiency disease(HA)
  source 1..20: mol_type = other DNA
ggtagcgagtcaataacgat

SEQ ID NO: 16 - DNA, 550 bp, synthetic construct
  misc_feature 1..550: note = Synthetic DNA containing portion of human gene encoding for Sickle Cell Anemia
  source 1..550: mol_type = other DNA
actcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctc

SEQ ID NO: 17 - DNA, 20 bp, synthetic construct
  misc_feature 1..20: note = Synthetic DNA for SgRNA seq.1 against Sickle cell Anemia (SCA)
  source 1..20: mol_type = other DNA
gtaacggcagacttctcctc

SEQ ID NO: 18 - DNA, 20 bp, synthetic construct
  misc_feature 1..20: note = Synthetic DNA for SgRNA seq.2 against Sickle cell Anemia (SCA)
  source 1..20: mol_type = other DNA
gtaacggcagacttatccac

SEQ ID NO: 19 - DNA, 20 bp, synthetic construct
  misc_feature 1..20: note = Synthetic DNA for double mismatch at HBB locus 1
  source 1..20: mol_type = other DNA
gtaacggcagacttctcaac

SEQ ID NO: 20 - DNA, 20 bp, synthetic construct
  misc_feature 1..20: note = Synthetic DNA for double mismatch at HBB locus 2
  source 1..20: mol_type = other DNA
gtaacggcagacttctacac

SEQ ID NO: 21 - DNA, 20 bp, synthetic construct
  misc_feature 1..20: note = Synthetic DNA for double mismatch at HBB locus 3
  source 1..20: mol_type = other DNA
gtaacggcagacttcaccac

SEQ ID NO: 22 - DNA, 20 bp, synthetic construct
  misc_feature 1..20: note = Synthetic DNA for double mismatch at HBB locus 4
  source 1..20: mol_type = other DNA
gtaacggcagacttatccac

SEQ ID NO: 23 - DNA, 20 bp, synthetic construct
  misc_feature 1..20: note = Synthetic DNA for double mismatch at HBB locus 5
  source 1..20: mol_type = other DNA
gtaacggcagactactccac

SEQ ID NO: 24 - DNA, 20 bp, synthetic construct
  misc_feature 1..20: note = Synthetic DNA for double mismatch at HBB locus 6
  source 1..20: mol_type = other DNA
gtaacggcagacatctccac

SEQ ID NO: 25 - DNA, 20 bp, synthetic construct
  misc_feature 1..20: note = Synthetic DNA for double mismatch at HBB locus 7
  source 1..20: mol_type = other DNA
gtaacggcagaattctccac

SEQ ID NO: 26 - DNA, 20 bp, synthetic construct
  misc_feature 1..20: note = Synthetic DNA for double mismatch at HBB locus 8
  source 1..20: mol_type = other DNA
gtaacggcagccttctccac

SEQ ID NO: 27 - DNA, 20 bp, synthetic construct
  misc_feature 1..20: note = Synthetic DNA for double mismatch at HBB locus 9
  source 1..20: mol_type = other DNA
gtaactgcagacttctccac

SEQ ID NO: 28 - DNA, 20 bp, synthetic construct
  misc_feature 1..20: note = Synthetic DNA for double mismatch at HBB locus 10
  source 1..20: mol_type = other DNA
gtaaaggcagacttctccac

SEQ ID NO: 29 - DNA, 20 bp, synthetic construct
  misc_feature 1..20: note = Synthetic DNA for double mismatch at HBB locus 11
  source 1..20: mol_type = other DNA
gtaccggcagacttctccac

SEQ ID NO: 30 - DNA, 20 bp, synthetic construct
  misc_feature 1..20: note = Synthetic DNA for double mismatch at HBB locus 12
  source 1..20: mol_type = other DNA
gtcacggcagacttctccac

SEQ ID NO: 31 - DNA, 20 bp, synthetic construct
  misc_feature 1..20: note = Synthetic DNA for double mismatch at HBB locus 13
  source 1..20: mol_type = other DNA
gaaacggcagacttctccac

SEQ ID NO: 32 - DNA, 20 bp, synthetic construct
  misc_feature 1..20: note = Synthetic WT sgRNA sequence for human HBB locus
  source 1..20: mol_type = other DNA
gtaacggcagacttctcctc

SEQ ID NO: 33 - DNA, 20 bp, synthetic construct
  misc_feature 1..20: note = Synthetic sgRNA sequence with mismatches at PAM proximal 2 and 6 positions
  source 1..20: mol_type = other DNA
gtaacggcagacttatccac

SEQ ID NO: 34 - DNA, 20 bp, synthetic construct
  misc_feature 1..20: note = Synthetic DNA sequence for HBB mismatched target 1
  source 1..20: mol_type = other DNA
gaagaaaagtctgccgttac

SEQ ID NO: 35 - DNA, 20 bp, synthetic construct
  misc_feature 1..20: note = Synthetic DNA sequence for HBB mismatched target 2
  source 1..20: mol_type = other DNA
gaggagaagtctgccattgc

SEQ ID NO: 36 - DNA, 72 bp, synthetic construct
  misc_feature 1..72: note = sgRNA with 1 mismatch for Glycogen Storage disease type I SNV and 2 mismatch for wild type sequence at 6th and 2nd position
  source 1..72: mol_type = other DNA
taatacgactcactataggccatgccatgggaacaacgtttcagttgcgccgaaaggcgctctgtaatcatt

SEQ ID NO: 37 - DNA, 24 bp, synthetic construct
  misc_feature 1..24: note = PCR primer for Glycogen storage disease type I
  source 1..24: mol_type = other DNA
gcataggagatggaatttgttaaa

SEQ ID NO: 38 - DNA, 22 bp, synthetic construct
  misc_feature 1..22: note = TCCCTGACTCTGAGGTTTATCC
  source 1..22: mol_type = other DNA
tccctgactctgaggtttatcc

SEQ ID NO: 39 - DNA, 72 bp, synthetic construct
  misc_feature 1..72: note = sgRNA with 1 mismatch for Glanzman ThRombasthenia SNV and 2 mismatch for wild type sequence at 6th and 2nd position
  source 1..72: mol_type = other DNA
taatacgactcactatagcagcccccagtccgcctcggtttcagttgcgccgaaaggcgctctgtaatcatt

SEQ ID NO: 40 - DNA, 19 bp, synthetic construct
  misc_feature 1..19: note = PCR primer for Glanzmann thrombasthenia disease
  source 1..19: mol_type = other DNA
acagaggcccacagcacac

SEQ ID NO: 41 - DNA, 20 bp, synthetic construct
  misc_feature 1..20: note = PCR primer for Glanzmann thrombasthenia disease
  source 1..20: mol_type = other DNA
cctgggactgtgaatggtct

SEQ ID NO: 42 - DNA, 72 bp, synthetic construct
  misc_feature 1..72: note = sgRNA with 1 mismatch for X-Linked myotubular myopathy SNV and 2 mismatch for wild type sequence at 6th and 2nd position
  source 1..72: mol_type = other DNA
taatacgactcactataggcaaatgtcaagacaggcagtttcagttgcgccgaaaggcgctctgtaatcatt

SEQ ID NO: 43 - DNA, 22 bp, synthetic construct
  misc_feature 1..22: note = PCR primer for X-linked myotubular myopathy disease
  source 1..22: mol_type = other DNA
cctctctgagaaacttggcatt

SEQ ID NO: 44 - DNA, 26 bp, synthetic construct
  misc_feature 1..26: note = PCR primer for X-linked myotubular myopathy disease
  source 1..26: mol_type = other DNA
tgtcaattccactagtagagacatca

SEQ ID NO: 45 - DNA, 72 bp, synthetic construct
  misc_feature 1..72: note = sgRNA with 1 mismatch for Hemophilia A(FactoR VIII) deficiency SNV and 2 mismatch for wild type sequence at 6th and 2nd position
  source 1..72: mol_type = other DNA
taatacgactcactataggtagcgagtcagtgacgatgtttcagttgcgccgaaaggcgctctgtaatcatt

SEQ ID NO: 46 - DNA, 25 bp, synthetic construct
  misc_feature 1..25: note = PCR primer for hereditory FactoR VIII deficiency disease
  source 1..25: mol_type = other DNA
gcaggactgatgatagttaattcag

SEQ ID NO: 47 - DNA, 20 bp, synthetic construct
  misc_feature 1..20: note = PCR primer for hereditory FactoR VIII deficiency disease
  source 1..20: mol_type = other DNA
gagtgaaggcctccaaagca

SEQ ID NO: 48 - DNA, 72 bp, synthetic construct
  misc_feature 1..72: note = sgRNA sequence for IVC against plasmid containing HBB sequence
  source 1..72: mol_type = other DNA
taatacgactcactatagtaacggcagacttctcctcgtttcagttgcgccgaaaggcgctctgtaatcatt

SEQ ID NO: 49 - DNA, 20 bp, synthetic construct
  misc_feature 1..20: note = PCR primer for 228bp HBB locus amplicon
  source 1..20: mol_type = other DNA
tctccacatgcccagtttct

SEQ ID NO: 50 - DNA, 20 bp, synthetic construct
  misc_feature 1..20: note = PCR primer for 228bp HBB locus amplicon
  source 1..20: mol_type = other DNA
agtcagggcagagccatcta

SEQ ID NO: 51 - DNA, 72 bp, synthetic construct
  misc_feature 1..72: note = sgRNA with 2 mismatches with Respect to HBB locus sgRNA keeping mismatch at 2nd and 3Rd position of sgRNA
  source 1..72: mol_type = other DNA
taatacgactcactatagtaacggcagacttctcaacgtttcagttgcgccgaaaggcgctctgtaatcatt

SEQ ID NO: 52 - DNA, 72 bp, synthetic construct
  misc_feature 1..72: note = sgRNA with 2 mismatches with Respect to HBB locus sgRNA keeping mismatch at 2nd and 4th position of sgRNA
  source 1..72: mol_type = other DNA
taatacgactcactatagtaacggcagacttctacacgtttcagttgcgccgaaaggcgctctgtaatcatt

SEQ ID NO: 53 - DNA, 72 bp, synthetic construct
  misc_feature 1..72: note = sgRNA with 2 mismatches with Respect to HBB locus sgRNA keeping mismatch at 2nd and 5th position of sgRNA
  source 1..72: mol_type = other DNA
taatacgactcactatagtaacggcagacttcaccacgtttcagttgcgccgaaaggcgctctgtaatcatt

SEQ ID NO: 54 - DNA, 72 bp, synthetic construct
  misc_feature 1..72: note = sgRNA with 2 mismatches with Respect to HBB locus sgRNA keeping mismatch at 2nd and 6th position of sgRNA
  source 1..72: mol_type = other DNA
taatacgactcactatagtaacggcagacttatccacgtttcagttgcgccgaaaggcgctctgtaatcatt

SEQ ID NO: 55 - DNA, 72 bp, synthetic construct
  misc_feature 1..72: note = sgRNA with 2 mismatches with Respect to HBB locus sgRNA keeping mismatch at 2nd and 7th position of sgRNA
  source 1..72: mol_type = other DNA
taatacgactcactatagtaacggcagactactccacgtttcagttgcgccgaaaggcgctctgtaatcatt

SEQ ID NO: 56 - DNA, 72 bp, synthetic construct
  misc_feature 1..72: note = sgRNA with 2 mismatches with Respect to HBB locus sgRNA keeping mismatch at 2nd and 8th position of sgRNA
  source 1..72: mol_type = other DNA
taatacgactcactatagtaacggcagacatctccacgtttcagttgcgccgaaaggcgctctgtaatcatt

SEQ ID NO: 57 - DNA, 72 bp, synthetic construct
  misc_feature 1..72: note = sgRNA with 2 mismatches with Respect to HBB locus sgRNA keeping mismatch at 2nd and 9th position of sgRNA
  source 1..72: mol_type = other DNA
taatacgactcactatagtaacggcagaattctccacgtttcagttgcgccgaaaggcgctctgtaatcatt

SEQ ID NO: 58 - DNA, 72 bp, synthetic construct
  misc_feature 1..72: note = sgRNA with 2 mismatches with Respect to HBB locus sgRNA keeping mismatch at 2nd and 10th position of sgRNA
  source 1..72: mol_type = other DNA
taatacgactcactatagtaacggcagccttctccacgtttcagttgcgccgaaaggcgctctgtaatcatt

SEQ ID NO: 59 - DNA, 72 bp, synthetic construct
  misc_feature 1..72: note = sgRNA with 2 mismatches with Respect to HBB locus sgRNA keeping mismatch at 2nd and 15th position of sgRNA
  source 1..72: mol_type = other DNA
taatacgactcactatagtaactgcagacttctccacgtttcagttgcgccgaaaggcgctctgtaatcatt

SEQ ID NO: 60 - DNA, 72 bp, synthetic construct
  misc_feature 1..72: note = sgRNA with 2 mismatches with Respect to HBB locus sgRNA keeping mismatch at 2nd and 16th position of sgRNA
  source 1..72: mol_type = other DNA
taatacgactcactatagtaaaggcagacttctccacgtttcagttgcgccgaaaggcgctctgtaatcatt

SEQ ID NO: 61 - DNA, 72 bp, synthetic construct
  misc_feature 1..72: note = sgRNA with 2 mismatches with Respect to HBB locus sgRNA keeping mismatch at 2nd and 17th position of sgRNA
  source 1..72: mol_type = other DNA
taatacgactcactatagtaccggcagacttctccacgtttcagttgcgccgaaaggcgctctgtaatcatt

SEQ ID NO: 62 - DNA, 72 bp, synthetic construct
  misc_feature 1..72: note = sgRNA with 2 mismatches with Respect to HBB locus sgRNA keeping mismatch at 2nd and 18th position of sgRNA
  source 1..72: mol_type = other DNA
taatacgactcactatagtcacggcagacttctccacgtttcagttgcgccgaaaggcgctctgtaatcatt

SEQ ID NO: 63 - DNA, 72 bp, synthetic construct
  misc_feature 1..72: note = sgRNA with 2 mismatches with Respect to HBB locus sgRNA keeping mismatch at 2nd and 19th position of sgRNA
  source 1..72: mol_type = other DNA
taatacgactcactatagaaacggcagacttctccacgtttcagttgcgccgaaaggcgctctgtaatcatt

SEQ ID NO: 64 - DNA, 20 bp, synthetic construct
  misc_feature 1..20: note = N gene crRNA sequence feluda
  source 1..20: mol_type = other DNA
gtggcagtacgtttttgccg

SEQ ID NO: 65 - DNA, 56 bp, synthetic construct
  misc_feature 1..56: note = SARS_CoV2 N gene crRNA Forword primer oligo for IVT
  source 1..56: mol_type = other DNA
taatacgactcactatagtggcagtacgtttttgccggtttcagttgctgaattat

SEQ ID NO: 66 - DNA, 56 bp, synthetic construct
  misc_feature 1..56: note = SARS_CoV2 N gene crRNA reverse primer oligo for IVT
  source 1..56: mol_type = other DNA
ataattcagcaactgaaaccgagggaatttaaggtcttctatagtgagtcgtatta

SEQ ID NO: 67 - DNA, 20 bp, synthetic construct
  misc_feature 1..20: note = S gene crRNA3 feluda
  source 1..20: mol_type = other DNA
gacattacaccatgttcttt

SEQ ID NO: 68 - DNA, 56 bp, synthetic construct
  misc_feature 1..56: note = SARS_CoV2 S gene crRNA Forward primer oligo for IVT feluda
  source 1..56: mol_type = other DNA
taatacgactcactatagacattacaccatgttctttgtttcagttgctgaattat

SEQ ID NO: 69 - DNA, 56 bp, synthetic construct
  misc_feature 1..56: note = SARS_CoV2 S gene crRNA reverse primer oligo for IVT feluda
  source 1..56: mol_type = other DNA
ataattcagcaactgaaacctaattacaaatgaatctgctatagtgagtcgtatta

SEQ ID NO: 70 - RNA, 39 nt, synthetic construct
  misc_feature 1..39: note = synthetic chimeric guide RNA part 1
  misc_difference 1..20: note = n is a, c, g, or u
  source 1..39: mol_type = other RNA
nnnnnnnnnnnnnnnnnnnngtttcagttgctgaattat

SEQ ID NO: 71 - RNA, 61 nt, synthetic construct
  misc_feature 1..61: note = Syntehtic chimerric guide RNA part 2
  source 1..61: mol_type = other RNA
gtaattaatgctctgtaatcatttaaaagtattttgaacggacctctgtttgacacgtctg

SEQ ID NO: 72 - RNA, 39 nt, synthetic construct
  misc_feature 1..39: note = Phosphorothioate modification between positions 1 and 2, and 37 and 38
  modified_base 1: mod_base = OTHER; note = Nucleotide with phosphorothioate linkage
  modified_base 37: mod_base = OTHER; note = Nucleotide with phosphorothioate linkage
  source 1..39: mol_type = other RNA
gacattacaccatgttctttgtttcagttgctgaattat

SEQ ID NO: 73 - RNA, 61 nt, synthetic construct; identical to SEQ ID NO: 71
  misc_feature 1..61: note = Phosphorothioate modification between positions 1 and 2, and position 59 and 60. FAM modification at 3' position.
  modified_base 1: mod_base = OTHER; note = Nucleotide with phosphorothioate linkage
  modified_base 59: mod_base = OTHER; note = Nucleotide with phosphorothioate linkage
  modified_base 61: mod_base = OTHER; note = Fluorescein modified nucleotide
  source 1..61: mol_type = other RNA

SEQ ID NO: 74 - DNA, 106 bp, synthetic construct
  misc_feature 1..106: note = Nucletide sequence corresponding to sgRNA for a representative locus
  misc_difference 1..20: note = n is a, c, g, or t
  source 1..106: mol_type = other DNA
nnnnnnnnnnnnnnnnnnnnagggttttagagctagaaatagcaagttaaaataaggctagtccgttatcaacttgaaaaagtggcaccgagtcggtgcttttttt

SEQ ID NO: 75 - DNA, 106 bp, synthetic construct
  misc_feature 1..106: note = nucleotide sequence corresponding to a representative example of sgRNA
  source 1..106: mol_type = other DNA
gtaacggcagacttctcctcagggttttagagctagaaatagcaagttaaaataaggctagtccgttatcaacttgaaaaagtggcaccgagtcggtgcttttttt

SEQ ID NO: 76 - DNA, 96 bp, synthetic construct
  misc_feature 1..96: note = Nucleotide sequence corresponding to representative sgRNA for HBB
  misc_difference 1..20: note = n is a, c, g, or t
  source 1..96: mol_type = other DNA
nnnnnnnnnnnnnnnnnnnnagggtttcagttgcgccgaaaggcgctctgtaatcatttaaaagtattttgaacggacctctgtttgacacgtctg

SEQ ID NO: 77 - DNA, 96 bp, synthetic construct
  misc_feature 1..96: note = Nucleotide corresponding to representative sgRNA for HBB locus
  source 1..96: mol_type = other DNA
gtaacggcagacttctcctcagggtttcagttgcgccgaaaggcgctctgtaatcatttaaaagtattttgaacggacctctgtttgacacgtctg

SEQ ID NO: 78 - DNA, 99 bp, synthetic construct
  misc_feature 1..99: note = Nucleotide corresponding to representative sgRNA sequence for SpCas9
  source 1..99: mol_type = other DNA
gtaacggcagacttatccacagggttttagagctagaaatagcaagttaaaataaggctagtccgttatcaacttgaaaaagtggcaccgagtcggtgc

SEQ ID NO: 79 - RNA, 39 nt, synthetic construct
  misc_feature 1..39: note = Synthetic representative single guide RNA for FnCas9
  source 1..39: mol_type = other RNA
ggtccaccaaacgtaatgcggtttcagttgctgaattat

SEQ ID NO: 80 - DNA, 96 bp, synthetic construct; identical to SEQ ID NO: 76
  misc_feature 1..96: note = Generic FnCas9 sgRNA sequence for disease detection with one mismatch at positions 16 /17 /18 /19 bp away from PAM
  misc_difference 1..20: note = n is a, c, g, or t
  source 1..96: mol_type = other DNA

SEQ ID NO: 81 - DNA, 20 bp, Helicobacter pylori
  source 1..20: mol_type = unassigned DNA
gaggagaagtctgccgttac

SEQ ID NO: 82 - DNA, 20 bp, synthetic construct
  misc_feature 1..20: note = Description of Artificial Sequence: Synthetic oligonucleotide
  source 1..20: mol_type = other DNA
gtaacggcagacttatccac

SEQ ID NO: 83 - DNA, 20 bp, synthetic construct
  misc_feature 1..20: note = Description of Artificial Sequence: Synthetic oligonucleotide
  source 1..20: mol_type = other DNA
gtggagaagtctgccgttac

SEQ ID NO: 84 - DNA, 40 bp, synthetic construct
  misc_feature 1..40: note = Description of Artificial Sequence: Synthetic oligonucleotide
  source 1..40: mol_type = other DNA
gtggagaagtctgccgttaccacctattcagacggcaatg

SEQ ID NO: 85 - DNA, 20 bp, Helicobacter pylori
  source 1..20: mol_type = unassigned DNA
gacggaaagaccccgtggac

SEQ ID NO: 86 - DNA, 20 bp, synthetic construct
  misc_feature 1..20: note = Description of Artificial Sequence: Synthetic oligonucleotide
  source 1..20: mol_type = other DNA
gacgggaagaccccgtggac

SEQ ID NO: 87 - DNA, 20 bp, synthetic construct
  misc_feature 1..20: note = Description of Artificial Sequence: Synthetic oligonucleotide
  source 1..20: mol_type = other DNA
gacggagagaccccgtggac

SEQ ID NO: 88 - DNA, 20 bp, synthetic construct
  misc_feature 1..20: note = Description of Artificial Sequence: Synthetic oligonucleotide
  source 1..20: mol_type = other DNA
gtccacggggtcttcccggc

SEQ ID NO: 89 - DNA, 20 bp, synthetic construct
  misc_feature 1..20: note = Description of Artificial Sequence: Synthetic oligonucleotide
  source 1..20: mol_type = other DNA
gtccacggggtctctccggc

SEQ ID NO: 90 - DNA, 40 bp, synthetic construct
  misc_feature 1..40: note = Description of Artificial Sequence: Synthetic oligonucleotide
  source 1..40: mol_type = other DNA
gaggagaagtctgccgttacctcctcttcagacggcaatg

SEQ ID NO: 91 - DNA, 40 bp, synthetic construct
  misc_feature 1..40: note = Description of Artificial Sequence: Synthetic oligonucleotide
  source 1..40: mol_type = other DNA
gaggagaagtctgccattacctcctcttcagacggcaatg

SEQ ID NO: 92 - DNA, 40 bp, synthetic construct; identical to SEQ ID NO: 90
  misc_feature 1..40: note = Description of Artificial Sequence: Synthetic oligonucleotide
  source 1..40: mol_type = other DNA

SEQ ID NO: 93 - DNA, 40 bp, synthetic construct
  misc_feature 1..40: note = Description of Artificial Sequence: Synthetic oligonucleotide
  source 1..40: mol_type = other DNA
gaggagaagtctgccgttaccacctattcagacggcaatg

(19) United States
(12) Patent Application Publication
(10) Pub. No.: US 2026/0193700 A1
(43) Pub. Date: Jul. 9, 2026

(54) METHOD AND KIT FOR DETECTION OF POLYNUCLEOTIDE

(71) Applicant: Council of Scientific & Industrial Research, New Delhi (IN)

(72) Inventors: Debojyoti Chakraborty, Delhi (IN); Souvik Maiti, Delhi (IN); Mohammad Azhar, Delhi (IN); Rhythm Phutela, Delhi (IN); Namrata Sharma, Delhi (IN); Dipanjali Sinha, Delhi (IN); Saumya Sharma, Delhi (IN); Arpit Mishra, Delhi (IN); Asgar Hussain Ansari, Delhi (IN)

(21) Appl. No.: 19/558,186

(22) Filed: Mar. 5, 2026

Related U.S. Application Data
(62) Division of application No. 17/781,726, filed on Dec. 1, 2020.

(30) Foreign Application Priority Data
Dec. 2, 2019 (IN) 201911049432
Mar. 27, 2020 (IN) 202011013418

(51) Int. Cl. C12Q 1/686; C07K 14/195; C12Q 1/6883; C12Q 1/70

(52) U.S. Cl. CPC C12Q 1/686; C07K 14/195; C12Q 1/6883; C12Q 1/701

(57) ABSTRACT

The invention describes a kit for detection of a target polynucleotide using a CRISPR effector system that comprises CAS9 from Francisella novicida, a synthetic sgRNA and a detection scheme based on binding and subsequent enzymatic cleavage of the target polynucleotide. The invention also describes a method for detection of a target polynucleotide using the kit. The kit can be applied to both pathogenic and non-pathogenic polynucleotides and can be used to distinguish polynucleotides different by a single mismatch without the need for sequencing. The kit can also be used for detection of COVID-19. The kit is economical, easy to assemble and provides a robust and rapid readout that can be appropriately adapted for point of care applications.

### SEQUENCE LISTING

The instant application contains a Sequence Listing which has been submitted electronically in XML format and is hereby incorporated by reference in its entirety. Said XML copy, created on Mar. 5, 2026, is named 134839-00171 SL.xml and is 135,511 bytes in size.

### FIELD OF INVENTION

The present invention relates to a kit for detection of a target polynucleotide using a CRISPR effector system comprising a CAS9 protein from Francisella novicida, and a single guide RNA. The present invention also relates to a method for detection of target polynucleotide.

### BACKGROUND OF THE INVENTION

A large number of pathogenic diseases in humans are either caused by or associated with nucleic acid variations. Detecting these variations with high precision can allow disease diagnosis, suitable healthcare interventions and prediction of disease outcome. Additionally, they also find use in molecular biology applications. In the past, several methods have been described that can detect nucleic acid variations with varying degrees of efficacy in terms of speed, accuracy, complexity and cost [Li, Y., Li, S., Wang, J. & Liu, G. CRISPR/Cas Systems towards Next-Generation Biosensing. Trends in Biotechnology (2019); Terns, M. P. CRISPR-Based Technologies: Impact of RNA-Targeting Systems. Biesecker, L. G. & Green, R. C. Diagnostic clinical genome and exome sequencing. N. Engl. J. Med. (2014); Nayak, S., Blumenfeld, N. R., Laksanasopin, T. & Sia, S. K. Point-of-Care Diagnostics: Recent Developments in a Connected Age. Analytical Chemistry (2017)].

Further, emergence of diseases caused by novel pathogenic organisms also creates a need for a method of detecting the pathogenic organisms with high specificity. 2019 Novel Coronavirus (2019-nCoV or SARS-CoV-2) is such a virus recently identified as the cause of an outbreak of respiratory illness (Coronavirus disease 2019, COVID-19) with an increasing number of patients with severe symptoms and deaths. To monitor the presence of SARS-CoV-2 and to prevent its spread, it is highly important to detect infection as early and as fast as possible with a sensitive, reliable test, not only in the clinic, but also in remote locations, without the need for laboratory equipment.

While most of the low-cost techniques require critical design parameters and suffer from poor sensitivity and specificity issues, the more precise sequencing-based detection platforms often require significant time to assemble in addition to being unsuitable for large scale point-of-care (POC) applications due to costs for instrumentation and diagnostic experimentation. Currently, the available method for detecting the presence of the coronavirus in a sample is through quantitative or semi quantitative reverse transcription polymerase chain reaction (RT PCR). Although such a method is widely used and has proven effective for disease diagnosis, it suffers from limitations including need for specialized equipment, trained workforce, and operational costs.

Based on the method available in the art, there is a need for a method for detection of pathogenic nucleotides that is selective, sensitive, reliable, and easily integrated in different settings around the world. Such a test must be simple, cost-effective, portable, able to be mass-produced, and easy to use in low resource settings so that vital clinical decisions can be made in a significantly short period of time.

Clustered regularly interspaced short palindromic repeats (CRISPR)/CRISPR-associated (Cas) were originally discovered to act as immunity defense mechanisms against foreign pathogens in prokaryotic cells (Mojica et al. (2005) J. of Molecular Evolution 60:174-182). Cas9, a protein for the type II CRISPR/Cas system, was found to exhibit DNA cleavage activity. The nuclease activity of Cas9 can be guided by a CRISPR RNA complementary to a targeted sequence of DNA in the genome and a trans-activating CRISPR RNA that forms a complex with Cas9 (Jinek et al. (2012) Science 337:816-821). The CRISPR/Cas system has been modified for diagnosis of diseases. However, systems and methods known in art fail to provide the activity and specificity necessary for the purpose of confirmed diagnosis. The Cas9 from Streptococcus pyogenes shows variable levels of off targeting due to tolerance of mismatches predominantly in the “nonseed” region in the sgRNA, wherever these are encountered in the genome (P. D. Hsu et al., DNA targeting specificity of RNA-guided Cas9 nucleases. Nat. Biotechnology 31, 827-832 (2013). These off targeting due to tolerance of mismatch leads to misdiagnosis, specifically in case of genetic diseases caused by single point mutation. Therefore, there is a need in the art for an improved CRISPR/Cas system having very low tolerance for mismatch for confirming diagnosis of disease.

### OBJECTIVES OF THE INVENTION

An objective of the present invention is to provide a kit for detection of a target polynucleotide using a CRISPR effector system comprising a CAS9 protein from Francisella novicida, and a single single guide RNA.

Another objective of the present invention is to provide a kit for detection of a single nucleotide variant of a polynucleotide using a CRISPR effector system comprising a CAS9 protein from Francisella novicida, and a single single guide RNA.

Yet another objective of the present invention is to provide a kit for detection of SARS-CoV2 using a CRISPR effector system comprising a CAS9 protein from Francisella novicida, and a single single guide RNA.

Still another objective of the present invention is to provide a method for detection of a target polynucleotide using the CRISPR system comprising a CAS9 protein and a single single guide RNA (sgRNA) that can specifically target nucleic acid molecules.

Yet another objective of the present invention is to utilize the method to distinguish between two nucleic acid molecules that are different by at least a single nucleotide. Another objective of the present invention is to provide a method for detection of coronavirus using the CRISPR system comprising a CAS9 protein and a single single guide RNA (sgRNA).

Still another objective of the invention is to use the kit as a point of care (POC) application for quick detection of target polynucleotide from patient samples.

### SUMMARY OF INVENTION

Accordingly, the present invention provides a kit for detection of a target polynucleotide comprising of
a. Reverse and forward primers;
b. dNTPS;
c. PCR buffer;
d. DNA polymerase;
e. FnCas9 protein having SEQ ID NO. 2;
f. single guide RNA;
g. CRISPR-CAS9 Reaction buffer.

In an embodiment of the invention it provides a kit, wherein the single guide RNA is having sequence selected from the group consisting of SEQ ID NOS. 32, 33, 36, 39, 42, 45, 48, 51-64, 67, 76, 77, 79, 80.

In an embodiment of the invention it provides a kit, wherein the primers are selected from the sequences comprising of sequences having SEQ ID NOS. 37, 38, 40, 41, 43, 44, 46, 47, 49, 50, 65, 66, 68, 69.

In an embodiment of the invention it provides a kit, wherein the PCR buffer for Recombinase polymerase amplification comprises RPA Rehydration buffer and Magnesium Acetate.

In an embodiment of the invention it provides a kit, wherein the kit optionally comprises reverse transcriptase.

In an embodiment of the invention it provides a kit, wherein the reaction buffer comprises of buffers selected from the group comprising of HEPES, KCl, DTT, glycerol and MgCl₂.

In an embodiment of the invention it provides a kit, wherein the primers are labelled with biotin.

In an embodiment of the invention it provides a kit, wherein the FnCas9 protein complexed with a single guide RNA is labelled with a chemical group including, but not limited to, Fluorescein Amidite (FAM).

In an embodiment of the invention it provides a kit, wherein the cas9 protein is isolated from Francisella novicida.

In an embodiment of the invention it provides a kit, wherein the Fncas9 protein with a fluorophore tag is having SEQ ID NO: 3.

In an embodiment of the invention it provides a kit, wherein the kit optionally comprises a reaction tube, a dipstick buffer and a paper strip.

In an embodiment of the invention it provides a kit, wherein the dipstick buffer is Tris buffered saline.

In an embodiment of the invention it provides a kit, wherein the paper strip is made of nitrocellulose membrane coated with biotin-ligand and polyclonal (goat) digoxigenin antibody or polyclonal (rabbit) anti-FITC antibody in gold conjugate.

In an embodiment of the invention it provides a kit, wherein the method for detection of a target polynucleotide comprising:
(a) isolating a polynucleotide;
(b) providing FnCas9 protein with single guide RNA;
(c) providing primers having sequence selected from the group consisting of SEQ ID NOS. 37, 38, 40, 41, 43, 44, 46, 47, 49, 50, 65, 66, 68, 69;
(d) amplifying the isolated polynucleotide of step (a) using Recombinase Polymerase Amplification or PCR with forward and reverse primers obtained in step (c) to obtain a product;
(e) incubating the product obtained in step (d) with a FnCas9 ribonucleoprotein complex obtained in step (b) in a reaction tube comprising a reaction buffer for a time period in the range of 10 to 20 minutes at a temperature in the range of 35 to 37° C.; and
(f) visualizing the results obtained in step (e) by a method selected from the group consisting of agarose gel electrophoresis, lateral flow detection and Fluorescence detection.

In an embodiment of the invention it provides a kit, wherein the target polynucleotide is a DNA or a RNA.

In an embodiment of the invention it provides a kit, wherein the target polynucleotide is a pathogenic or non-pathogenic DNA or RNA.

In an embodiment of the invention it provides a kit, wherein the target polynucleotide is a SARS-COV2.

In an embodiment of the invention it provides a kit, wherein the target polynucleotide is a single nucleotide variant related to a disease selected from the group consisting of, but not limited to, sickle cell anemia, Glanzmann's Thrombasthenia, Glycogen Storage Disease Type I, Hemophilia A, X-linked myotubular myopathy.

In an embodiment of the invention it provides a kit, wherein the optionally Reverse transcription is carried out after step (a) when the polynucleotide is RNA.

In an embodiment of the invention it provides a kit, wherein the FnCas9 protein is isolated from Francisella novicida having SEQ ID NO: 2 and 3.

In an embodiment of the invention it provides a kit, wherein the single guide RNA has two parts having SEQ ID NO. 70 and 71.

In an embodiment of the invention it provides a kit, wherein the single guide RNA is having sequence selected from the group consisting of SEQ ID NOS. 32, 33, 36, 39, 42, 45, 48, 51-64, 67, 76, 77, 79, 80.

In an embodiment of the invention it provides a kit, wherein the method of lateral flow detection comprises,
a) adding a paper strip and dipstick buffer to the reaction tube after step 14(c); and
b) incubating the tube obtained in step (a) for a period in the range of 5 to 10 minutes to observe a visible band on the paper strip, wherein the presence of the band on the paper strip confirms the presence of target polynucleotide.

In an embodiment of the invention it provides a kit, wherein the FnCas9 ribonucleoprotein complex comprising a cas9 protein from Francisella novicida having the amino acid sequence as set forth in SEQ ID NO.: 2, and a single guide RNA having SEQ ID NO. 79.

In an embodiment of the invention it provides a kit, wherein the single guide RNA or its corresponding sequence is selected from the group consisting of SEQ ID NOS.: 5, 6, 8, 9, 11, 12, 14, 15, 17, 18, 19-36, 39, 42, 45, 48, 76, 77, 79, 80.

In an embodiment of the invention it provides a kit, wherein the corresponding DNA targeting region in SARS-CoV2 has a sequence as set forth in SEQ ID NO.: 64, 67.

In an embodiment of the invention it provides a kit, wherein the system for detecting diseases caused by single nucleotide variants (SNVs) wherein the system consisting of:
a. Cas 9 effector protein;
b. Sg RNA;
c. PCR primers for amplification of a target;
d. Reverse transcriptase if the sequence is an RNA sequence and amplification enzymes like a Taq polymerase;
e. Lateral flow assay strip with its buffer.

In an embodiment of the invention it provides a kit, wherein the single guide RNA is selected from the group consisting of SEQ ID NOS 5, 6, 8, 9, 11, 12, 14, 15, 17, 18, 19-36, 39, 42, 45, 48, 64, 67, 76, 77, 79, 80.

In an embodiment of the invention it provides a kit, wherein the primers are selected from the group consisting of SEQ ID NOS. 37, 38, 40, 41, 43, 44, 46, 47, 49, 50, 65, 66, 68, 69.

In an embodiment of the invention it provides a kit, wherein the Cas9 protein is isolated from Francisella novicida.

In an embodiment of the invention it provides a kit, wherein the Cas9 protein is fluorescent labeled.

In an embodiment of the invention it provides a kit, wherein the target nucleic acid is selected from DNA and RNA having SEQ ID NOS. 4, 7, 10, 13, 16.

In an embodiment of the invention it provides use of the system for detecting diseases selected from the group comprising of Genetic disorders like sickle cell anemia, Glanzmann's Thrombasthenia, Glycogen Storage Disease Type I, Hemophilia A, X-linked myotubular myopathy and Pathogenic diseases caused by microbes selected from the group comprising of SARS-CoV2, SARS CoV1, Helicobacter pylori infection.

### BRIEF DESCRIPTION OF THE DRAWINGS

FIG. 1. Protein sequence of dead FnCas9 (A) and dFnCas9-GFP (B).

FIG. 2. Schematic showing components of a representative CAS9 Ribonucleoprotein complex.

FIG. 3. In vitro cleavage of a target polynucleotide with CAS9.

FIG. 4. (A-D) Binding affinity measurements of CAS9 with mismatched polynucleotide.

FIG. 5. Isothermal amplification of a target using Recombination Polymerase Amplification (RPA).

FIG. 6. In vitro cleavage of product obtained by RPA reaction.

FIG. 7. Schematic showing the adaptation of the system for cleavage-free detection.

FIG. 8. Schematic for fluorescence-based detection of sickle cell anemia mutation using FELUDA. Error bars represent SD (3 independent experiments).

FIG. 9. Quantification of fluorescence readout for cleavage-free detection for WT and homozygous Sickle cell anemia mutation.

FIG. 10. Outcome of FELUDA on WT, SCT or SCA substrates. Error bars represent s.e.m (n=3 independent replicates, student t-test p values are shown).

FIG. 11. Upper panel shows schematic of FELUDA for identifying carriers of SCA mutation. Lower panel shows blinded FELUDA results in a mixed cohort of individuals (n=49, one-way ANOVA p value is shown).

FIG. 12. CAS9 based discrimination of sickle cell anemia variant in DNA I.

FIG. 13. CAS9 based discrimination of sickle cell anemia variant in DNA II.

FIG. 14. Gel based detection of Heterozygous SNPs in DNA.

FIG. 15. Outline of lateral flow assay using FELUDA showing positions of control and test bands.

FIG. 16. Adaptation of chimeric gRNA for FELUDA. Cleavage outcomes with different lengths of overlap between crRNA and tracrRNA are shown.

FIG. 17. Pipeline of FELUDA based detection for SARS-CoV-2 infection in samples obtained from patients. Individual steps are depicted.

FIG. 18. FELUDA for COVID-19 diagnosis with time duration of individual steps are shown.

FIG. 19. Plot showing the regions of nCoV-2 RNA genome tested for FELUDA, successful regions represented in red.

FIG. 20. sgRNA design strategy ensuring inclusivity of all known strains of SARS-CoV2 and no cross reactivity with other respiratory viruses like Influenza virus.

FIG. 21. LOD of FELUDA in purified target RNA. Top panel shows representative LFA readout on strips, bottom panel shows Fluorescent intensity ratios. Error bars s.e.m. (n=3 independent experiments).

FIG. 22. Representative LFA strips showing optimized temperature and primer concentrations for FELUDA for COVID-19.

FIG. 23. Plot showing distribution of LFA intensities (y-axis) with dilution of patient RNA (Ct value, x-axis) for S gene and N gene. Correlation between the two are represented as R values.

FIG. 24. Strong reproducibility between repeated FELUDA (RNP incubation and readout) on the same positive or negative sample is shown.

FIG. 25. One pot RT-RPA FELUDA. Top panel shows minimum requirements, bottom panel shows outcome for 2 representative samples.

FIG. 26. FELUDA readouts are semiquantitative. Correlation between Ct values (E gene) and TOPSE values are shown (n=27).

FIG. 27. One gene FELUDA on clinical samples (x axis) showing distribution of TOPSE values (y axis). Analyzed results represented on the right.

FIG. 28. Optimization of 2 gene FELUDA. (A) LFA readout of samples with a repeat FELUDA (N gene) and corresponding Ct values for E and ORF genes. IC, internal controls. Values in black and green are from 1^{st} run and 2^{nd} run respectively. Red asterisk denotes samples which became FELUDA positive on a repeat run. (B) Samples classified as negative in one qPCR run (shown in black) but showing Ct values of E gene or E/ORF both genes on a second run (shown in green). LFA strips of such samples are marked with red asterisk.

FIG. 29. Two gene FELUDA on clinical samples (x axis) showing distribution of TOPSE values (y axis). Analyzed results represented on the right.

FIG. 30. Samples identified by N and S genes in 2 gene FELUDA. Ct value distribution is shown on x-axis, TOPSE values depicted on y-axis.

FIG. 31. On-body RT-RPA-FELUDA, left figure shows variation of temperature in different zones of the body marked in red dots and corresponding RPA-FELUDA for a synthetic RNA fragment as starting material. Center panel shows representative FELUDA using on body RT-RPA from samples incubated in two different parts of the body. Right panel represents minimum requirements for FELUDA using on-body RT-RPA.

FIG. 32. Strategy for discrimination of substrates differing by single mismatch using FnCas9. Presence of 2 mismatches (marked in red and green) at defined positions on the sgRNA prevents the enzyme from binding to the target leading to different binding and cleavage outcomes.

FIG. 33. Left panel shows positions of mismatches sgRNAs containing two mismatches at different positions along their lengths. Representative in-vitro cleavage outcomes on wild type (WT) or sickle cell anemia (SCA) substrates (4.1 kb) are shown on the right. Cleavage with FnCas9 produces 2 products (2.3 kb and 1.8 kb). Red dotted box denotes the sgRNA showing negligible cleavage for WT substrate and maximum cleavage for SCA substrate.

FIG. 34. FELUDA design can universally discriminate SNVs across diverse targets. Representative gel image showing synthetic targets containing either WT or pathogenic SNVs. FELUDA can distinguish between the two variants.

FIG. 35. Binding experiments using Microscale Thermophoresis showing interaction of FnCas9 with substrates with 1 mismatch (MM) on the left and 2 mismatches on the right. Values are expressed as fraction bound protein (y-axis) with respect to varying concentrations of purified DNA substrate (Molar units, M, x-axis). Error bars represent SEM (2 independent experiments).

FIG. 36. Top, strategy for detection of SNVs with an in-built PAM (PAM-mer) in primer sequence. Bottom, closely related strains of H. Pylori showing the mutations they are associated with in red.

FIG. 37. Two different sgRNAs, A2142G and A2143G can produce distinct cleavage outcomes with their respective substrates (left). FELUDA based detection of A2143G genotype of H Pylori from gut biopsy of a patient (right). Cleavage outcomes with synthetic substrates for A2143G and A2142G are also shown.

FIG. 38. Representative agarose gel showing GFP substrate cleaved with sgRNAs harboring mismatches at PAM distal 16-19th position. WT sgRNA (0) shows successful cleavage.

FIG. 39. Representative gel image showing no cleavage of HBB substrate with sgRNA containing 1 mismatch (16th position, PAM distal). Binding experiments using Microscale Thermophoresis showing high binding affinity with no mismatches to substrate and negligible binding affinity when 1 mismatch at 16th position (PAM distal) is present (MM, mismatches). Values are expressed as fraction bound protein (y-axis) with respect to varying concentrations of purified DNA substrate (Molar units, M, x-axis). Error bars represent SEM (2 independent experiments).

FIG. 40. Sanger sequencing confirming the allelic variants obtained from 4 individuals and FELUDA design based on placing mismatch at 16th position enables detection of variants. Negligible cleavage is seen with G variant, maximum cleavage is seen with C variant and intermediate cleavage seen with G/C variant.

FIG. 41. Representative gel images showing successful cleavage of substrates at different temperatures (10° C.-50° C.).

FIG. 42. Representative image of a substrate cleavage reaction using FnCAS9 RNP complex. Complete match to intended substrate (COVID-19) produces cleavage of bound substrate (3 kb) to distinct signatures of 2.2 kb and 0.9 kb. Two different sgRNA forms (dual or single) produce similar readouts. ON represents specific sgRNAs while OFF represents non-specific sgRNAs. HBB (human haemoglobin locus targeting sgRNAs) is used as a positive control. Control represents substrate without RNP and is not cleaved.

FIG. 43. Binding affinity experiments using Microscale Thermophoresis showing interaction of dSpCas9-GFP, dSpCas9-HF1-GFP and dFnCas9-GFP with substrates with 0 (□) or 2 (∘) mismatch (MM). Values are expressed as fraction bound protein (y-axis) with respect to varying concentrations of purified DNA substrate (Molar units, M, x-axis). Error bars represent SEM (2 independent experiments).

FIG. 44. Representative images showing differences between unmodified (IVT, in vitro transcribed) vs Phosphorothioate modified (syn, synthetic) gRNA (Genes 1 and 2) [stored at −20 and after undergoing multiple freeze thaw processes].

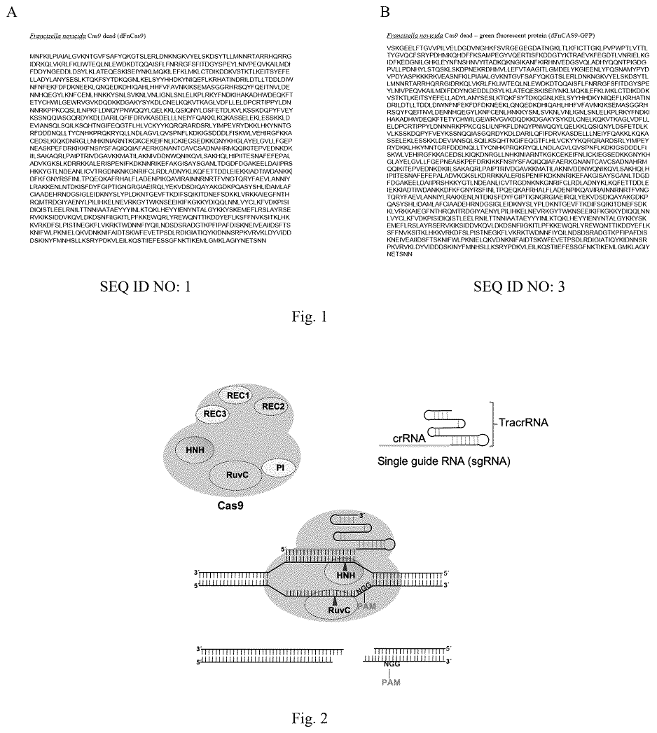

### BRIEF DESCRIPTION OF SEQUENCE LISTING

1. SEQ ID NO.1—Protein sequence of recombinant FnCas9 from Francisella novicida.
2. SEQ ID NO.2—Protein sequence of recombinant dead FnCas9 (dFnCas9) from Francisella novicida.
3. SEQ ID NO.3—Protein sequence of recombinant dEnCas 9 from Francisella novicida coupled with green Fluorescent protein.
4. SEQ ID NO.4—synthetic DNA containing portion of human gene encoding for Glycogen storage disease type I.
5. SEQ ID NO. 5—Synthetic DNA for SgRNA seq.1 against Glycogen storage disease type I.
6. SEQ ID NO.6—Synthetic DNA for SgRNA seq.2 against Glycogen storage disease type I.
7. SEQ ID NO.7—Synthetic DNA containing portion of human gene encoding for Glanzmann thrombasthenia.
8. SEQ ID NO.8—Synthetic DNA for SgRNA seq.1 against Glanzmann thrombasthenia.
9. SEQ ID NO.9—Synthetic DNA for SgRNA seq.2 against Glanzmann thrombasthenia.
10. SEQ ID NO.10—Synthetic DNA containing portion of human gene encoding for X-linked myotubular myopathy.
11. SEQ ID NO.11—Synthetic DNA for SgRNA seq.1 against X-linked myotubular myopathy.
12. SEQ ID NO.12—Synthetic DNA for SgRNA seq.2 against X-linked myotubular myopathy.
13. SEQ ID NO.13—Synthetic DNA containing portion of human gene encoding for Hereditary Factor VIII deficiency disease (HA).
14. SEQ ID NO.14—Synthetic DNA for SgRNA seq 1 against Hereditary factor VIII deficiency disease (HA).
15. SEQ ID NO.15—Synthetic DNA for SgRNA seq 2 against Hereditary factor VIII deficiency disease (HA).
16. SEQ ID NO.16—Synthetic DNA containing portion of human gene encoding for Sickle Cell Anemia.
17. SEQ ID NO.17—Synthetic DNA for SgRNA seq.1 against Sickle cell Anemia (SCA).
18. SEQ ID NO.18—Synthetic DNA for SgRNA seq.2 against Sickle cell Anemia (SCA).
19. SEQ ID NO.19—Synthetic DNA for double mismatch at HBB locus 1.
20. SEQ ID NO.20—Synthetic DNA for double mismatch at HBB locus 2.
21. SEQ ID NO.21—Synthetic DNA for double mismatch at HBB locus 3.
22. SEQ ID NO.22—Synthetic DNA for double mismatch at HBB locus 4.
23. SEQ ID NO.23—Synthetic DNA for double mismatch at HBB locus 5.
24. SEQ ID NO.24—Synthetic DNA for double mismatch at HBB locus 6.
25. SEQ ID NO.25—Synthetic DNA for double mismatch at HBB locus 7.
26. SEQ ID NO.26—Synthetic DNA for double mismatch at HBB locus 8.
27. SEQ ID NO.27—Synthetic DNA for double mismatch at HBB locus 9.
28. SEQ ID NO.28—Synthetic DNA for double mismatch at HBB locus 10.
29. SEQ ID NO.29—Synthetic DNA for double mismatch at HBB locus 11.
30. SEQ ID NO.30—Synthetic DNA for double mismatch at HBB locus 12.
31. SEQ ID NO.31—Synthetic DNA for double mismatch at HBB locus 13.
32. SEQ ID NO.32—Synthetic WT SgRNA Sequence for human HBB locus.
33. SEQ ID NO.33—Synthetic WT SgRNA Sequence with mismatches at PAM proximal 2 and 6 positions.
34. SEQ ID NO.34—Synthetic DNA sequence for HBB mismatched target 1.
35. SEQ ID NO.35—Synthetic DNA sequence for HBB mismatched target 2.
36. SEQ ID NO.36—SgRNA with 1 mismatch for Glycogen Storage disease type I SNV and 2 mismatch for wild type sequence at 6^{th} and 2^{nd} position.
37. SEQ ID NO.37—PCR Primer for Glycogen storage disease type I.
38. SEQ ID NO.38—PCR Primer for Glycogen storage disease type I.
39. SEQ ID NO. 39—SgRNA with 1 mismatch for Glanzmann Thrombasthenia SNV and 2 mismatch for wild type sequence at 6^{th} and 2^{nd} position.
40. SEQ ID NO.40—PCR Primer for Glanzmann thrombasthenia disease.
41. SEQ ID NO.41—PCR Primer for Glanzmann thrombasthenia disease.
42. SEQ ID NO.42—SgRNA with mismatch for X-linked myotubular myopathy SNV and 2 mismatch for wild type sequence at 6^{th} and 2^{nd} position.
43. SEQ ID NO.43—PCR Primer for X-linked myotubular myopathy disease.
44. SEQ ID NO.44—PCR Primer for X-linked myotubular myopathy disease.
45. SEQ ID NO.45—SgRNA with 1 mismatch for Hemophilia A (Factor VIII) deficiency SNV and 2 mismatch for wild type sequence at 6^{th} and 2^{nd} position.
46. SEQ ID NO.46—PCR Primer for hereditary Factor VIII deficiency disease.
47. SEQ ID NO.47—PCR Primer for hereditary Factor VIII deficiency disease.
48. SEQ ID NO.48—SgRNA sequence for IVC against plasmid containing HBB sequence.
49. SEQ ID NO.49—PCR Primer for 228 bp HBB locus amplicon.
50. SEQ ID NO.50—PCR Primer for 228 bp HBB locus amplicon.
51. SEQ ID NO.51—SgRNA with 2 mismatches with respect to HBB locus SgRNA keeping mismatch at 2^{nd} and 3^{rd} position of SgRNA.
52. SEQ ID NO.52—SgRNA with 2 mismatches with respect to HBB locus SgRNA keeping mismatch at 2^{nd} and 4^{th} position of SgRNA.
53. SEQ ID NO.53—SgRNA with 2 mismatches with respect to HBB locus SgRNA keeping mismatch at 2^{nd} and 5^{th} position of SgRNA.
54. SEQ ID NO.54—SgRNA with 2 mismatches with respect to HBB locus SgRNA keeping mismatch at 2^{nd} and 6^{th} position of SgRNA.
55. SEQ ID NO.55—SgRNA with 2 mismatches with respect to HBB locus SgRNA keeping mismatch at 2^{nd} and 7^{th} position of SgRNA.
56. SEQ ID NO.56—SgRNA with 2 mismatches with respect to HBB locus SgRNA keeping mismatch at 2^{nd} and 8^{th} position of SgRNA.
57. SEQ ID NO.57—SgRNA with 2 mismatches with respect to HBB locus SgRNA keeping mismatch at 2^{nd} and 9^{th} position of SgRNA.
58. SEQ ID NO.58—SgRNA with 2 mismatches with respect to HBB locus SgRNA keeping mismatch at 2^{nd} and 10^{th} position of SgRNA.
59. SEQ ID NO.59—SgRNA with 2 mismatches with respect to HBB locus SgRNA keeping mismatch at 2^{nd} and 15^{th} position of SgRNA.
60. SEQ ID NO.60—SgRNA with 2 mismatches with respect to HBB locus SgRNA keeping mismatch at 2^{nd} and 16^{th} position of SgRNA.
61. SEQ ID NO.61—SgRNA with 2 mismatches with respect to HBB locus SgRNA keeping mismatch at 2^{nd} and 17^{th} position of SgRNA.
62. SEQ ID NO.62—SgRNA with 2 mismatches with respect to HBB locus SgRNA keeping mismatch at 2^{nd} and 18^{th} position of SgRNA.
63. SEQ ID NO.63—SgRNA with 2 mismatches with respect to HBB locus SgRNA keeping mismatch at 2^{nd} and 19^{th} position of SgRNA.
64. SEQ ID NO.64—N gene crRNA sequence FELUDA.
65. SEQ ID NO.65—SARS_CoV2 N gene crRNA Forward primer oligo for IVT.
66. SEQ ID NO.66—SARS_CoV2 N gene crRNA reverse primer oligo for IVT.
67. SEQ ID NO.67—S gene crRNA sequence FELUDA.
68. SEQ ID NO.68—SARS_CoV2 S gene crRNA Forward primer oligo for IVT feluda.
69. SEQ ID NO.69—SARS_CoV2 S gene crRNA reverse primer oligo for IVT feluda.
70. SEQ ID NO.70—Synthetic chimeric single guide RNA part 1.
71. SEQ ID NO.71—Synthetic chimeric single guide RNA part 2.
72. SEQ ID NO.72—Modified chimeric single guide RNA part 1.
73. SEQ ID NO.73—Modified chimeric single guide RNA part 2.
74. SEQ ID NO.74—Nucleotide sequence for sgRNA.
75. SEQ ID NO.75—Nucleotide sequence for sgRNA.
76. SEQ ID NO.76—Representative sgRNA for HBB locus.
77. SEQ ID NO.77—Representative sgRNA for HBB locus.
78. SEQ ID NO.78—Representative sgRNA for SpCas9.
79. SEQ ID NO.79—Representative sgRNA for FnCas9.
80. SEQ ID NO.80—Generic sgRNA for FnCas9 with one mismatch.

### ABBREVIATIONS USED

FELUDA: FNCAS9 Editor-Limited Uniform Detection Assay

### DETAILED DESCRIPTION OF INVENTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

A “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type DNA” is DNA that is naturally occurring in or endogenous to the organism.

Also as used herein, the terms “nucleic acid,” “nucleic acid molecule,” and “polynucleotide” refer to RNA or DNA that is linear or branched, single or double stranded, or a hybrid thereof. The term also encompasses RNA/DNA hybrids.

“Cas9” refers to a large group of endonucleases that catalyze the double stranded DNA cleavage in the CRISPR Cas system.

The CRISPR-Cas system does not require the generation of customized proteins to target specific sequences but rather a single Cas enzyme can be programmed by a short RNA molecule to recognize a specific DNA target. An exemplary CRISPR complex comprises a CRISPR enzyme complexed with a guide sequence hybridized to a target sequence within the target polynucleotide. The present invention provides methods for using one or more elements of a CRISPR system. The CRISPR complex of the invention provides an effective means for disease diagnosis.

The CRISPR effector system of the present invention comprises an effector protein and a single guide RNA designed to bind to corresponding target molecules. The CRISPR effector system of the present invention is used for distinguishing two nucleic acid molecules that differ by at least one nucleotide in their respective sequences. The present invention also provides a CRISPR effector system for rapidly identifying the COVID-19 nucleotide sequence in a sample for early diagnosis and preventive measures for community transmission.

In an embodiment of the present invention, target RNAs and/or DNAs may be amplified prior to activating the CRISPR effector protein. Any suitable RNA or DNA amplification technique may be used. The techniques include but are not limited to non-isothermal amplification PCR, isothermal PCR, multiple displacement amplification (MDA), rolling circle amplification (RCA), ligase chain reaction (LCR), or ramification amplification method (RAM).

In a preferred embodiment of isothermal PCR, recombinase polymerase amplification (RPA) reaction is used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. RPA reactions can also be used to amplify target RNA. The target RNA is first converted to cDNA using a reverse transcriptase, followed by second strand DNA synthesis, at which point the RPA reaction proceeds as outlined above.

In an embodiment, the present invention provides a detection system for detection of a target polynucleotide comprising of a CRISPR effector protein, a sgRNA molecule targeting a polynucleotide sequence, reagents to amplify the polynucleotide and a fluorescent or colorimetric detection system. The system comprises reagents for nucleic acid amplification. These include a pair of long primers, one of which contains a promoter sequence for an RNA polymerase. The two primers can be annealed and extended in a PCR reaction followed by transcription for producing sgRNAs.

The detection system consists of a CAS9 protein and a sgRNA consisting of two parts: a crRNA region that recognizes target polynucleotide and a tracrRNA region that is required for CAS9 protein binding and recruitment to target polynucleotide. The CAS9 protein can be from Francisella novicida or any other bacteria that show little to no affinity with polynucleotide harboring mismatches between crRNA and target. In a preferred embodiment of the present invention, the CAS9 protein is from Francisella novicida. The CAS9 protein can be engineered for inactivating its cleavage properties and additionally carrying fluorescent or biochemical tags to enable detection or purification of ribonucleoprotein complex (RNP complex) associated/dissociated from polynucleotide.

The detection system may consist of one or more mismatches incorporated in the crRNA region of the sgRNA to distinguish between two target polynucleotide molecules. These mismatches may be incorporated at defined positions with respect to the protospacer adjacent motif (PAM) sequence on the polynucleotide. The detection system distinguishes between targets on the basis of the differences in binding affinities to the individual polynucleotides that are different by at least one nucleotide. The CRISPR effector complex binds matched polynucleotide very efficiently while showing minimal binding at targets containing at least one mismatched nucleotide. The starting material for obtaining a target polynucleotide for CRISPR effector mediated cleavage may be genomic DNA of a subject. The starting material for obtaining a target polynucleotide for CRISPR effector mediated cleavage is a circular DNA or linear DNA. In a preferred embodiment of the present invention, the target polynucleotide to be detected can be COVID-19 RNA converted into complementary DNA (cDNA) prior to being used as a substrate for amplification.

In another embodiment of the present invention, the target polynucleotide may be amplified by Polymerase Chain Reaction (PCR) using primers specific to the target region. In a preferred embodiment, the conversion of RNA to DNA can be done using a Reverse Transcription (RT) reaction using appropriate enzymes. In a preferred embodiment, the DNA or cDNA target can be amplified using isothermal amplification method Recombinant Polymerase Amplification (RPA) using specific primers that can amplify the target polynucleotide at a single temperature.

In still another embodiment of the present invention, a synthetic PAM site may be introduced in one of the PCR primers to facilitate the detection of single nucleotide variants (SNVs) that do not have an adjacent PAM. In a preferred embodiment, the RNA can be reverse transcribed to cDNA with a PAM containing reverse primer and then a PCR step can be performed on the DNA resulting in the amplicon that can be targeted by CRISPR components.

In another embodiment of the present invention, the target to be detected can be isolated from body fluids of an individual organism. The bodily fluids are selected from the group consisting of saliva, sputum, serum, plasma, blood, pharyngeal, nasal/nasal pharyngeal and sinus secretions, urine, mucous, gastric juices, pancreatic juices, bone marrow aspirates, cerebral spinal fluid, feces, semen, products of lactation or menstruation, cervical secretions, vaginal fluid, tears and lymph.

In an embodiment of the present invention, the primers used for amplification are biotin-labelled to enable downstream product purification using streptavidin. The polynucleotide amplified by PCR may be purified before subjected to CRISPR effector system mediated cleavage to ensure externally added components of the PCR reaction do not hamper the cleavage reaction.

In another embodiment of the present invention, the target polynucleotide cleavage or binding reactions using purified effector protein and sgRNA can occur within an hour. The detection system can cleave polynucleotide matching the crRNA sequence completely and not cleave polynucleotide that do not match the crRNA sequence completely leading to a visually distinct pattern of DNA products resolved by agarose gel electrophoresis. The agarose gel electrophoresis pattern of polynucleotide cleavage can rapidly determine the presence of an SNV in a target incubated with the CRISPR effector complex.

In still another embodiment the amplified polynucleotide can be incubated with the CRISPR effector complex and a readout based on interaction of the complex with the polynucleotide can be obtained. The CAS9 protein used can be active (intact nuclease domains) or inactive (mutated nuclease domains). If an intact CAS9 protein is used, the target polynucleotide will be cleaved, and a distinct agarose gel electrophoresis signature will be obtained for COVID-19 sequence. The electrophoresis signature will be different if assayed with a sequence other than COVID-19. If an inactive CAS9 protein is used, the target polynucleotide will be bound (but not cleaved) if an sgRNA containing a perfectly matched sequence is used while not bound if an sgRNA with mismatches is used. A control non-targeting sgRNA can give an alternate signature thereby distinguishing COVID-19 from a different viral strain.

In another embodiment of the present invention, the agarose gel electrophoresis may be substituted by a system that can analyze bio-fragments generating a distinguishable pattern read out on a detection device. The device for analyzing the bio-fragments can be portable and field-deployable. Further, the inactive CAS9 protein can be fluorescently labelled so that a fluorescent readout can be obtained after interaction with the target polynucleotide. The fluorescent label can be added to the tracrRNA part of the dual sgRNA. The fluorescence detection system can be adapted to a lateral flow device such as a paper strip for rapid detection of COVID-19 identity or single nucleotide variant causing genetic diseases. The paper strip system can give a distinct band upon accumulation of biotin tagged substrate on a streptavidin line.

In yet another embodiment, the detection can be through a fluorescence reader based on the binding of CAS9 RNP and an immobilized substrate complex. The detection can be fluorometric based on fluorescence emitted by CAS9-RNP upon association or dissociation with its target polynucleotide without the need for cleavage.

The detection system can be simplified into a point-of-care device for rapid genotyping from patient samples. The detection system can be optimized for discriminating both homozygous and heterozygous SNVs. The detection system can be optimized for identifying carriers of a disease mutation.

In an embodiment of the present invention, the detection system can use synthetic sgRNAs containing chemical modifications. The chemical modifications can improve the binding of the sgRNA to its substrate and improve the overall detection efficiency. The detection system can use engineered protein that gives a more robust readout due to enhanced properties of DNA interrogation. The detection system can be converted into a kit capable of multiplexed detection of target polynucleotide.

In another embodiment of the present invention, the detection system can be used to generate a rapid readout of multiple point mutations in a sample. The detection system can be used to distinguish DNA or RNA from strains of pathogenic microorganisms different by at least 1 nucleotide.

In another embodiment of the present invention, the detection system can be used to identify a nucleotide at a given position in a polynucleotide sequence. The identity can be generated by binding affinity of the CAS9-RNP complex with the polynucleotide with position specific modifications in the sgRNA.

In the method for detection of a target polynucleotide of the present invention, the target polynucleotide is a DNA or RNA. The target polynucleotide can be a pathogenic or non-pathogenic DNA or RNA. In the preferred embodiment of the present invention, the target polynucleotide is a COVID-19 RNA. In another preferred embodiment, the target polynucleotide is a single nucleotide variant related to a disease selected from the group consisting of, but not limited to, sickle cell anemia, Glanzmann's Thrombasthenia, Glycogen Storage Disease Type I, Hemophilia A, X-linked myotubular myopathy.

For the detection of a target polynucleotide, the polynucleotide is isolated from bodily fluids selected from the group consisting of saliva, sputum, serum, plasma, blood, pharyngeal, nasal/nasal pharyngeal and sinus secretions, urine, mucous, gastric juices, pancreatic juices, bone marrow aspirates, cerebral spinal fluid, feces, semen, products of lactation or menstruation, cervical secretions, vaginal fluid, tears and lymph. In the preferred embodiment, the bodily fluid is blood. In another preferred embodiment, the bodily fluid is nasal/nasal pharyngeal and sinus secretions.

In an embodiment of the present invention, there is provided a method for detection of a target polynucleotide, wherein optionally Reverse transcription is carried out before step (b) when the polynucleotide is an RNA.

In the method for detection of a target polynucleotide of the present invention, the FnCas9 ribonucleoprotein complex comprises a cas9 protein from Francisella novicida, a single single guide RNA (sgRNA), In the single single guide RNA (sg RNA), the crRNA is specific towards the target polynucleotide. Further, the tracrRNA can be labeled with a chemical modification selected from the group consisting of amidites, biotin, streptavidin or digoxigenin. In the method for detection of a target polynucleotide of the present invention, the cas9 protein is catalytically inactive having the amino acid sequence as set forth in SEQ ID NO.: 2.

The present invention also provides a FnCas9 ribonucleoprotein complex comprising a cas9 protein from Francisella novicida which is catalytically inactive having the amino acid sequence as set forth in SEQ ID NO.: 2, and a single single guide RNA (sg RNA).

An embodiment of the present invention is FELUDA (FNCAS9 Editor-Limited Uniform Detection Assay), wherein FELUDA accurately genotypes carriers of single nucleotide variants. Although sickle cell trait (SCT) individuals are generally non-symptomatic, carrier screening is vital to prevent the spread of SCA in successive generations and is widely employed in SCA control programs in various parts of the world. Since FELUDA outcomes are reflected by binding to substrate molecules, it resulted in clearly distinguishable signatures between the SCA, SCT and WT DNA obtained from patient's saliva samples (FIG. 10). FELUDA is able to identify all three genotypes with 100% accuracy and the results perfectly matched with Sanger sequencing data generated on the same samples.

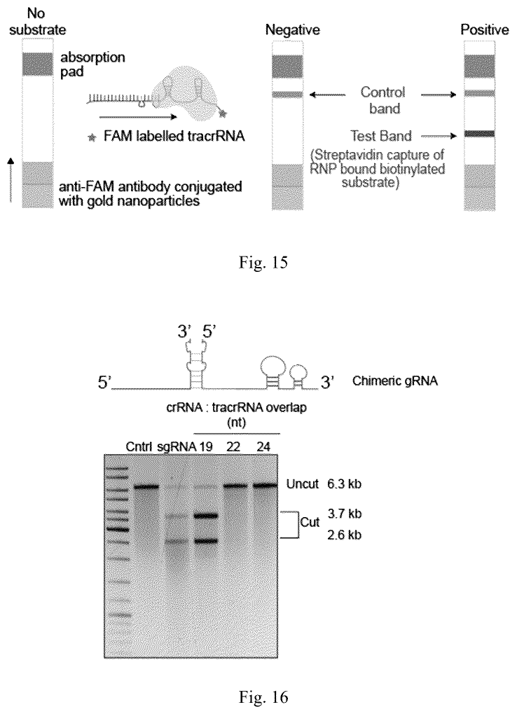

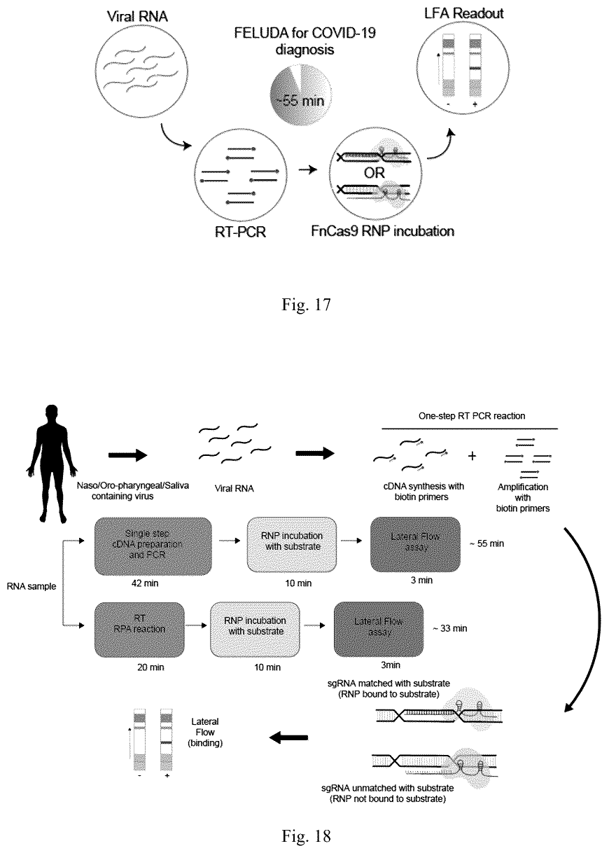

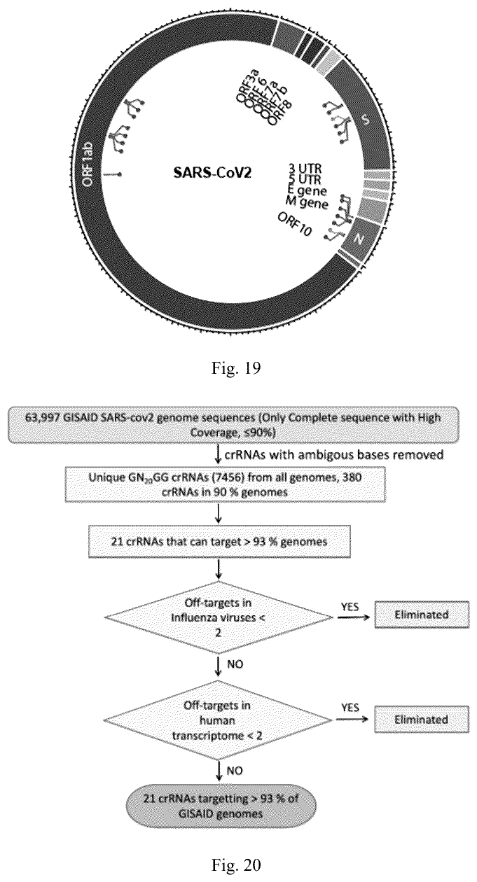

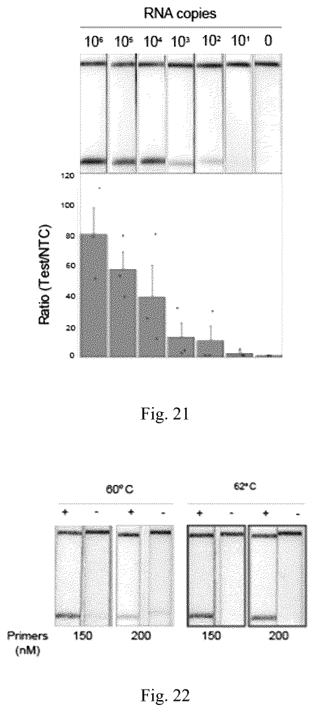

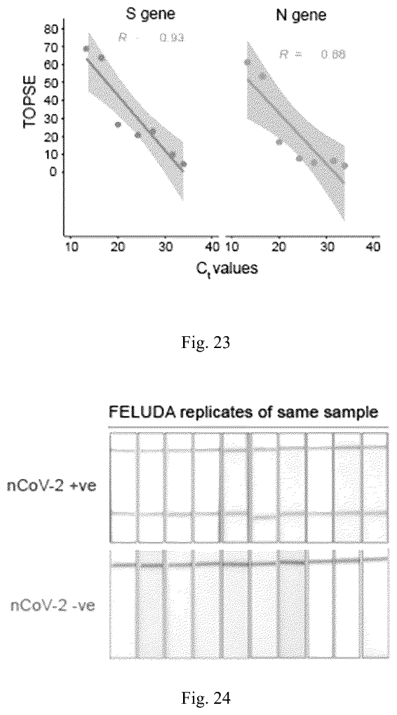

In the embodiment, FELUDA is repurposed as a lateral flow assay (LFA) for the detection of SARS-CoV-2 that is low-cost, does not need complex instrumentation, and is highly accurate in diagnosis. To enable such a diagnosis on commercially available paper strips, the chemistry of capturing RNP-bound biotinylated substrate molecules is enabled on a distinct test line of the paper strip using FAM labeled chimeric gRNA (FIG. 15, FIG. 16). Using an optimized single step Reverse Transcription-PCR protocol followed by FELUDA, an assay has been developed that can detect SARS-CoV-2 sequences from RNA samples within an hour (FIG. 17 and FIG. 18). Up to 21 targets were tested across the SARS-CoV-2 RNA genome and identified two regions (in the viral N and S genes) which are present at high copy numbers and reported negligible number of mutations in publicly available datasets (63,997, GISAID submissions till Sep. 7, 2020) (FIG. 19, FIG. 20). Through extensive optimization of PCR and reaction components, FELUDA reached a limit of detection (LOD) of ~10 copies of purified viral sequence (FIG. 21, FIG. 22). Upon gradual dilution of patient RNA, both FELUDA and qRT-PCR was able to detect samples till the same dilution range (FIG. 23).

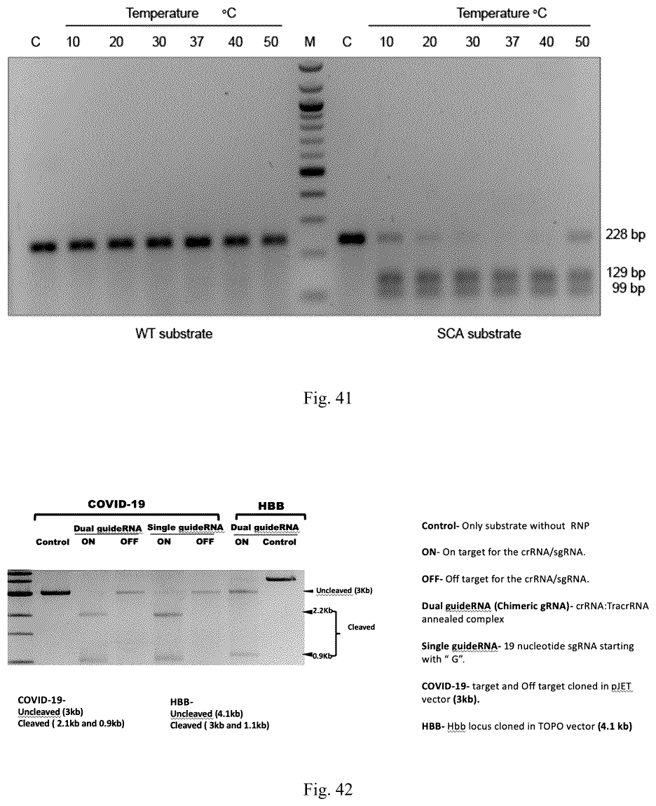

In the embodiment, FELUDA based detection can work robustly across a wide temperature range and up to 3 days post thawing of reaction components (at room temperature). Thus, field studies using FELUDA can be conducted in diverse climatic conditions and reaction components can be successfully used following cold chain transportation (FIG. 41).

### EXAMPLES

The following examples are given by way of illustration and therefore should not be construed to limit the scope of the invention.

### Example 1

CAS9 Protein and sgRNA Purification

The proteins used in the present invention were purified as reported in [Jinek, M. et al. A programmable dual-RNA-guided DNA endonuclease in adaptive bacterial immunity. Science (80) (2012)]. Briefly, plasmids for CAS9 from Francisella novicida were expressed in Escherichia coli Rosetta2 (DE3) (Novagen®). The protein expressing Rosetta2 (DE3) cells were cultured at 37° C. in LB medium (supplemented with 50 mg/l kanamycin) until OD₆₀₀ reached 0.6 and protein expression was induced by addition of 0.5 mM isopropyl β-D-thiogalactopyranoside (IPTG). The Rosetta2 (DE3) cells were further cultured at 18° C. overnight and harvested by centrifugation. The E. coli cells were resuspended in lysis buffer (20 mM Hepes, pH7.5, 250 mM NaCl, 5% glycerol, 1 mM DTT, 1 mM PMSF) supplemented with 1×PIC (Roche®), 100 ug/ml lysozyme and lysed by sonication and centrifuged. The lysate was affinity purified by Ni-NTA beads (Roche®) and the eluted protein (SEQ ID NO 1-3) was further purified by size-exclusion chromatography HiLoad® Superdex® 200 16/600 column (GE Healthcare™) in 20 mM HEPES pH 7.5, 150 mM KCl, 10% glycerol, 1 mM DTT, 10 mM MgCl₂. The concentrations of purified proteins were measured by Pierce BCA protein assay kit (Thermo Fisher Scientific®). The purified proteins were stored at −80° C. until further use.

In vitro transcribed sgRNA's were synthesized using MegaScript™ T7 Transcription kit's (Thermo Fisher Scientific®) using a T7 promoter containing template as substrates. IVT reactions were incubated overnight at 37° C. followed by NucAway spin column (Thermo Fisher Scientific®) purification. IVT sgRNAs were stored at −20° C. until further use. FIG. 2 shows the schematic representation of components of a representative CAS9 Ribonucleoprotein (RNP) complex.

### Example 2

In Vitro Cleavage Reactions of Target with CAS9 RNP Complex

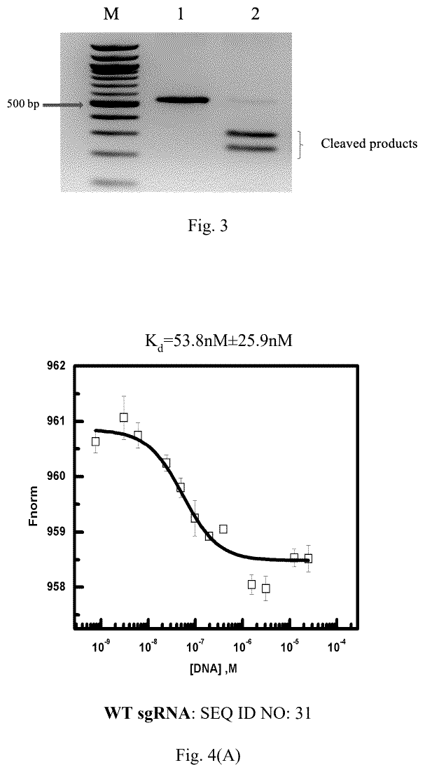

250 nM CAS9-sgRNA complex, (wherein the Cas9 could be one of the sequences in SEQ ID NO. 1-3, and a representative sgRNA sequence SEQ ID NO. 48) was reconstituted at 25° C. for 10 mins in reaction buffer (20 mM HEPES, pH7.5, 150 mM KCl, 1 mM DTT, 10% glycerol, 10 mM MgCl₂). 100 ng of PCR amplified substrate was incubated in reaction buffer with reconstituted RNP complex at 37° C. The reaction was then treated with 1 ul of 20 mg/ml Proteinase K in 15 μl reaction for 15 mins at 55° C. followed by the heat inactivation at 70° C. for 10 minutes. After then 1 ul of 20 mg/ml RNase treatment was given at 37° C. for 10 minutes, the reaction products were resolved in EtBr-stained 1.5% agarose gel, visualized by Syngene® Gel Doc. FIG. 3. shows In vitro cleavage of a target polynucleotide with CAS9 (SEQ ID NO. 1-3), which shows that FnCas9 can specifically identify and cleave DNA sequences that are targeted by sgRNA.

### Example 3

Binding Affinity of a Representative CAS9 Complex (FnCAS9) with Target sgRNAs And Mismatched sgRNAs

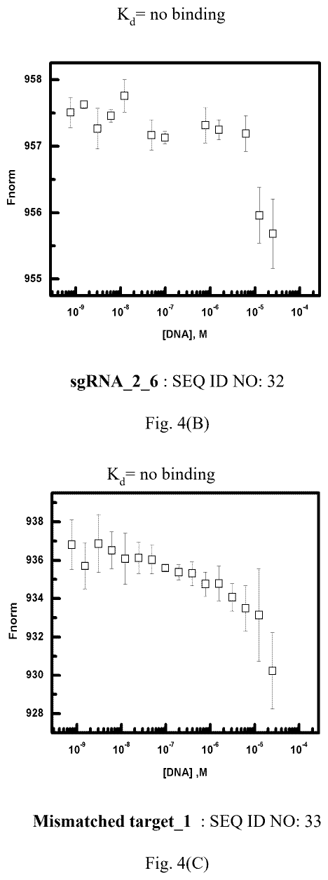

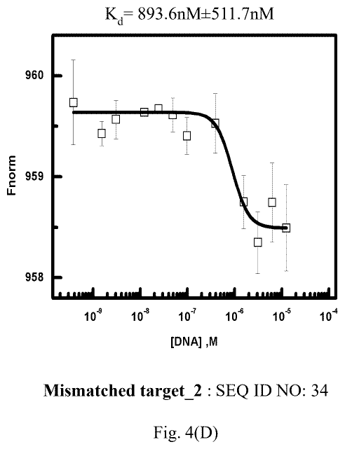

MST was performed as reported previously [Dong, D. et al. The crystal structure of Cpf1 in complex with CRISPR RNA. Nature (2016); Wienken, C. J., Baaske, P., Rothbauer, U., Braun, D. & Duhr, S. Protein-binding assays in biological liquids using microscale thermophoresis. Nat. Commun. (2010)]. Briefly, dFnCAS9-GFP (SEQ ID NO: 3) protein was complexed with PAGE purified respective IVT sgRNAs (purified by 12% Urea-PAGE). The binding affinities of the CAS9 proteins and sgRNA RNP complexes were calculated using Monolith NT. 115 (NanoTemper® Technologies GmbH, Munich, Germany). RNP complex (Protein: sgRNA molar ratio, 1:1) was reconstituted at 25° C. for 10 mins in reaction buffer (20 mM HEPES, pH7.5, 150 mM KCl, 1 mM DTT, 10 mM MgCl₂). HPLC purified 30 bp dsDNA (IDT) of different genomic loci with varying concentrations (ranging from 0.09 nM to 30 μM) were incubated with RNP complex at 37° C. temperature for 30 min in reaction buffer. The sample was loaded into NanoTemper® standard treated capillaries and measurements were performed at 25° C. using 20% LED power and 40% MST power. Data analyses were done using NanoTemper® analysis software. FIG. 4 shows the binding affinity of FnCas9 with WT (SEQ ID NO: 32) and mismatched substrates (SEQ ID NO: 33-35). A low dissociation constant was seen for SEQ ID NO: 32 while, no binding was seen for SEQ ID NO: 33 and SEQ ID NO:34 and a very high dissociation constant for SEQ ID NO:35, thereby the presence of mismatches between sgRNA and target sequence abolishes binding of FnCas9, confirming its very high specificity.

### Example 4

### RPA for Amplifying Target and its Subsequent Cleavage Using FnCAS9 Complex

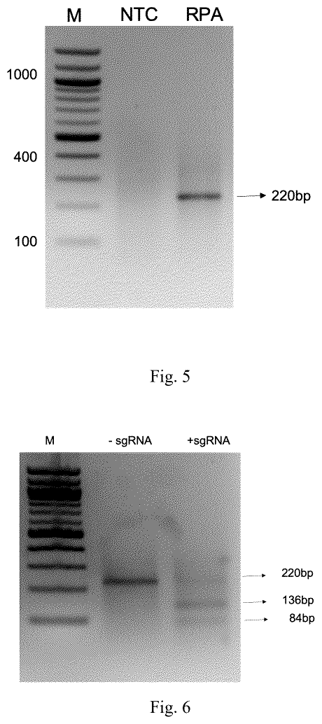

47.5 ul reaction mixes was reconstituted in 2.4 ul each of 10 μM stock of forward and reverse primers of Emx1 gene, 29.5 ul of Primer Free Rehydration buffer (TwistAmp® Basic kit), 3-5 ng of genomic DNA and remaining reaction volume was made up by Nuclease Free Water. The reaction was added to a TwistAmp® Basic reaction (supplied with the TwistAmp® Basic kit) and mixed well. 2.5 μl of 280 mM Magnesium Acetate (MgOAc) was added to start the reaction. The reaction was then incubated at 39° C. for 20 minutes and then the amplicons were cleaned up by the Qiagen® PCR clean up kit. The amplicon was resolved in EtBr-stained 2.5% agarose gel, visualized by Syngene® Gel Documentation apparatus. FIG. 5 shows the Isothermal amplification of a target using Recombination Polymerase Amplification (RPA).

250 nM CAS9-sgRNA complex was reconstituted at 25° C. for 10 mins in reaction buffer (20 mM HEPES, pH7.5, 150 mM KCl, 1 mM DTT, 10% glycerol, 10 mM MgCl₂). 100 ng of 220 bp EMX1 amplicon was incubated in reaction buffer with reconstituted RNP complex for 60 minutes at 37° C. The reaction was then treated with 1 ul of 20 mg/ml Proteinase K in 15 μl reaction for 15 mins at 55° C. followed by the heat inactivation at 70° C. for 10 minutes. After the 1 ul of 20 mg/ml RNase treatment at 37° C. for 10 minutes, the reaction products were resolved in EtBr-stained 2.5% agarose gel and visualized by Syngene® Gel Documentation apparatus. FIG. 6. In vitro cleavage of product obtained by RPA reaction, which confirms the activity of FnCas9 after RPA and maintains the specificity for binding and cleavage of target.

### Example 5

### Fluorescent Detection of Monogenic Variants

50 μL of Dynabeads™ MyOne™ Streptavidin C1 (Thermo Fisher Scientific®) were washed three times with 1 ml of buffer containing 50 mM TrisCl pH 7.5, 5 mM EDTA, 1M NaCl. 5 μL of the washed beads were incubated with 222 ng of 228 bp biotin labelled Hbb PCR amplicon (wild type and Sickle cell sample, SEQ ID NOs 49-50) for 30 minutes in 50 μL previously mentioned buffer. 200 nM (dFnCas9 GFP): 400 nM (sgRNA) RNP complex was reconstituted at 25° C. for 10 mins in 30 μL reaction buffer (20 mM HEPES, pH7.5, 150 mM KCl, 1 mM DTT, 10% glycerol, 10 mM MgCl₂). 30 μL of the reconstituted RNP complex {200 nM (dFnCas9-GFP, SEQ ID NO. 3): 400 nM (sgRNA)} was incubated with the streptavidin beads bound with biotin labelled PCR amplicon for 20 minutes. Unbound dFnCas9 GFP emission spectra (495 nm-555 nm) were measured after 20 minutes incubation excitation at 408 nm.

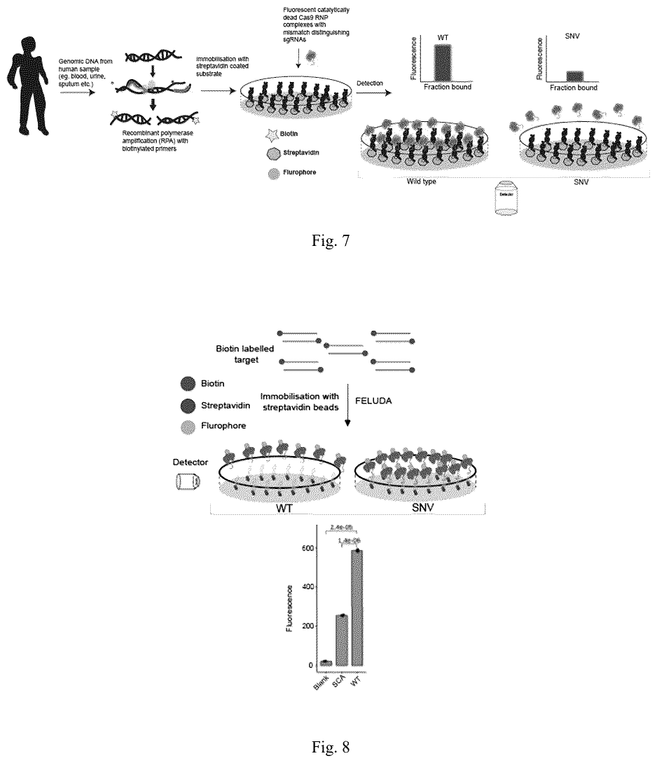

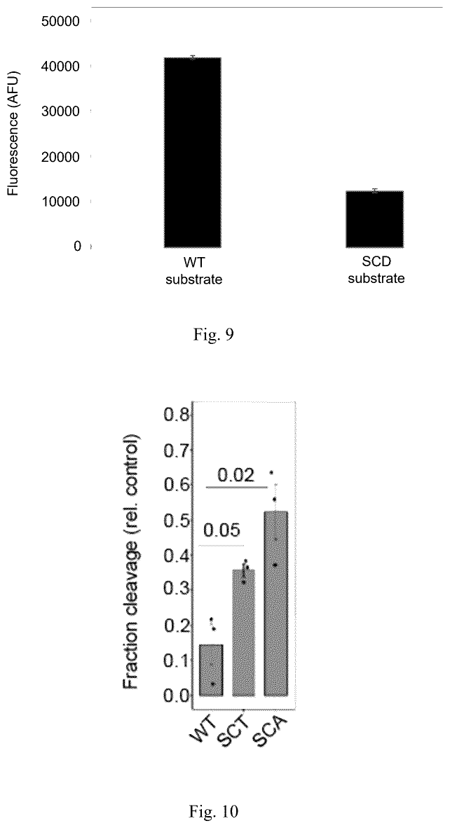

FIGS. 7 and 8 show the schematic representation showing the adaptation of the system for cleavage-free detection. FIG. 9 shows the quantification of fluorescence readout for cleavage-free detection for WT and homozygous Sickle cell anemia mutation. FnCas, thus could distinguish between the wt and scd substrates that have a single bp difference confirming the very high specificity of FnCas9, that can be measured using fluorescence values. It also demonstrated the successful binding-based detection of a single mismatch using FnCas9.

To enable robust readout and longer shelf-life, a chimeric sgRNA was developed in two parts with 19 nucleotide overlap (Part1 and Part 2 to be annealed to make full length sgRNA).

### Part 1:

(SEQ ID No: 70)
5′
NNNNNNNNNNNNNNNNNNNNGUUUCAGUUGCUGAAUUAU
3′

### Part 2:

(SEQ ID NO: 71)
5′-GUAAUUAAUGCUCUGUAAUCAUUUAAAAGUAUU
UUGAACGGACCUCUGUUUGACACGUCUG
3′

N corresponds to the nucleotide stretch in the target. An example is as follows:

(SEQ ID NO: 79)
5′-GGUCCACCAAACGUAAUGCGGUUUCAGUUGCUGAAUUAU-3′

The chimeric design performed similar to that of a full length sgRNA showing no loss of activity due to the split-sgRNA synthesis and also without any loss of specificity for 1 mismatch discrimination (FIG. 42).

### Example 6

### SCA FELUDA Detection Assay

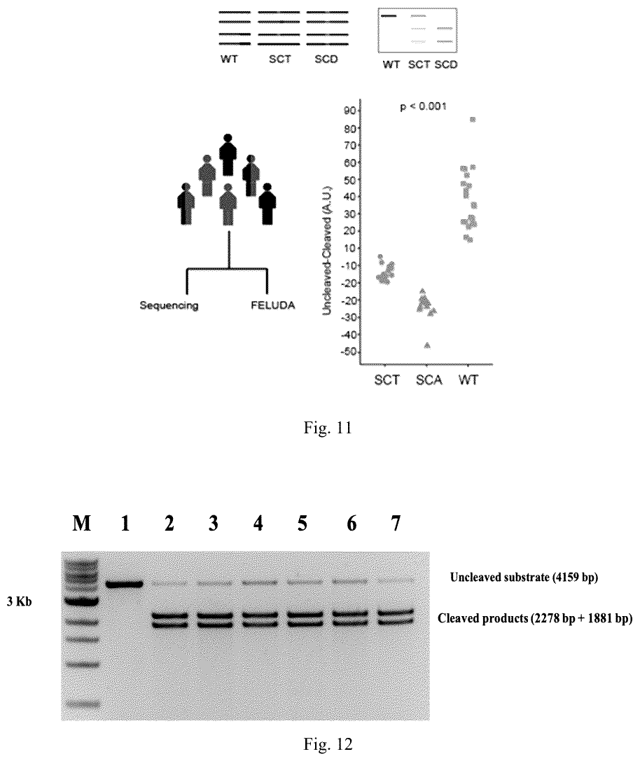

FELUDA accurately genotypes carriers of Mendelian variants. Although sickle cell trait (SCT) individuals are generally non-symptomatic, carrier screening is vital to prevent the spread of SCA (Sickle cell anemia) in successive generations and is widely employed in SCA control programs in various parts of the world. Since FELUDA outcomes are reflected by binding to substrate molecules, it resulted in clearly distinguishable signatures between the SCA, SCT and WT DNA obtained from patient's saliva samples (FIG. 10). To address the robustness of detecting the 3 genotype categories, a blinded experiment was performed using DNA obtained from 49 subjects with all three SCA genotypes from a CSIR-Sickle Cell Anaemia Mission Laboratory in Chhattisgarh, India. Remarkably, FELUDA identified all three genotypes with 100% accuracy and the results perfectly matched with Sanger sequencing data generated on same samples in a different laboratory (CSIR-Center for Cellular and Molecular Biology, Genome Research on Complex Diseases Lab) (FIG. 11).

Sequences containing WT, SCT and SCA were amplified using primers with/without 5′ biotinylation from genomic DNA extracted from saliva or blood samples. Detection via In vitro Cleavage (IVC): DNA or RNA converted to DNA were PCR amplified to be used as a substrate in in vitro cleavage assay. ~100 ng of purified DNA amplicon was incubated in reaction buffer (20 mM HEPES, pH7.5, 150 mM KCl, 1 mM DTT, 10% glycerol, 10 mM MgCl₂) along with reconstituted RNP complex (500 nM) at 37° C. for 30 minutes and cleaved products were visualized on agarose gel. Detection via Fluorescence Detection: DNA regions from Hbb locus (WT & SCA) were amplified using biotinylated primers. dFnCas9-GFP: sgRNA (180 nM: 540 nM) RNP complex was reconstituted at 25° C. for 10 mins in reaction buffer (20 mM HEPES, pH7.5, 150 mM KCl, 1 mM DTT, 10% glycerol, 10 mM MgCl₂). Meanwhile, 6 μL of the Dynabeads™ MyOne™ Streptavidin C1 (Thermo Fisher Scientific®) were prepared. Beads were incubated with 1 μM of biotinylated Hbb amplicon (WT & SCA) for 30 minutes in the reaction buffer. Further, dFnCas9-GFP RNP complex was incubated with the streptavidin bound PCR amplicon for 30 minutes. Emission spectra of unbound dFnCas9-GFP was measured using Monolith NT. 115 (NanoTemper® Technologies GmbH, Munich, Germany) under 60% excitation power in blue filter (465-490 nm excitation wavelength; 500-550 nm emission wavelength) with medium MST power.

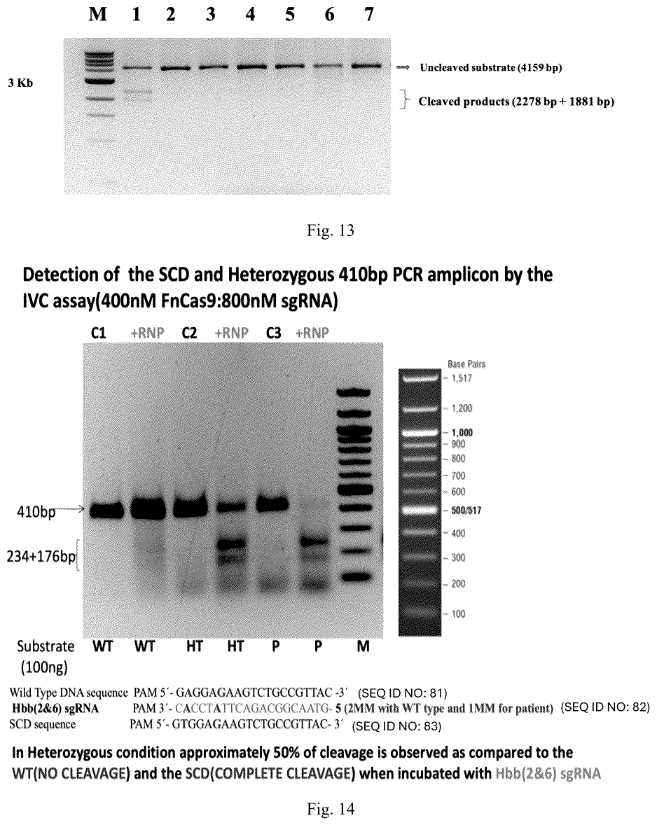

CAS9 based detection of Sickle cell anemia SNP in amplified patient (versus control) DNA leads to characteristic cleavage pattern in case of mutation and absence in case of wild type DNA as provided in FIG. 12. CAS9 based discrimination of Sickle cell anemia SNP in amplified patient (versus control) DNA leads to cleavage pattern in case of wild type DNA and absence in case of sickle cell mutation as provided in FIG. 13. Gel based detection of Heterozygous SNPs in DNA can be seen in FIG. 14 (SEQ ID NOs. 16-18, 32).

### Example 7

### Lateral Flow Detection of COVID-19

FELUDA was sought to be repurposed as a lateral flow assay (LFA) for the detection of SARS-CoV-2 that is low-cost, does not need complex instrumentation, and is highly accurate in diagnosis. To enable such a diagnosis on commercially available paper strips, the chemistry of capturing RNP-bound biotinylated substrate molecules was enabled on a distinct test line of the paper strip using FAM labeled chimeric gRNA (FIG. 15, FIG. 16). Using an optimized single step Reverse Transcription-PCR protocol followed by FELUDA, an assay was developed that can detect SARS-CoV-2 sequences from RNA samples within an hour (FIG. 17 and FIG. 18). Up to 21 targets were tested across the SARS-CoV-2 RNA genome and identified two regions (in the viral N and S genes) which are present at high copy numbers and reported negligible number of mutations in publicly available datasets (63,997, GISAID submissions till Sep. 7, 2020) (FIG. 19, FIG. 20). Through careful optimization of PCR and reaction components, FELUDA reached a limit of detection (LOD) of ~10 copies of purified viral sequence (FIG. 21, FIG. 22). Upon gradual dilution of patient RNA, both FELUDA and qRT-PCR was able to detect samples till the same dilution range (FIG. 23). Since visual detection can occasionally have an operator-bias, particularly when the signal is very faint, a smartphone app TOPSE (True Outcome Predicted via Strip Evaluation) was developed to assist detection by returning a predictive score based on background correction.

Biotin Labelled Amplification of the substrate: Amplification of the COVID 19 polynucleotide with the biotin labelled primers by RPA (TwistDx™) or Thermal Cycler (Reverse transcription 50° C. for 15 minute, Initial denaturation 95° C. for 1 min, 5 Pre-amplification cycles of 95° C. for 5 sec; 60° C. for 40 sec followed by 40 amplification cycles of 95° C. for 5 sec; 60° C. for 40 sec) was carried out. After the confirmation of amplicon, PCR clean-up was done (Qiagen®) followed by quantification of the product. 4 μM of the COVID 19 specific crRNA along with the FnCas9 FAM labelled tracrRNA was reconstituted in a buffer containing 100 mM NaCl, 50 mM Tris HCl pH 8 and 1 mM MgCl2 by placing the reaction at room temperature for 15 mins followed by heating at 95° C. for 5 minutes. For binding of Cas9 complex to substrate, 500 nM of the chimeric FAM labelled sgRNA and inactive FnCas9 was reconstituted in a buffer (20 mM HEPES, pH7.5, 150 mM KCl, 1 mM DTT, 10% glycerol, 10 mM MgCl2) at 25° C. for 10 minutes. Following this, 2.2 nM of the PCR product was incubated with the RNP complex for 15 minutes at 37° C. in a reaction tube. Then, dipstick buffer was added in the reaction and the strips were added into the reaction tube. Gold nanoparticles are bound on the strip. Streptavidin is bound on the test line in the strip which captures the gold nanoparticle bound FELUDA complex. Visible bands can be visualised after 5-10 minutes of the incubation. Intensity of the bands gets darker with time.

FIG. 15 shows the outline of lateral flow assay using FELUDA showing positions of control and test bands. FIG. 17 shows the pipeline of FELUDA based detection for SARS-CoV-2 infection in samples obtained from patients. Individual steps are depicted. FIG. 24 shows the strong reproducibility between repeated FELUDA (RNP incubation and readout) on the same positive or negative sample is shown. 10 replicates for SARS-CoV2 positive and negative samples are shown in this figure.

### Example 8

### FELUDA Limit of Detection (LOD)

Synthetic genomic Target for N gene was serially diluted (1:10, 7 times) from ~4×106 copies/μl to perform FELUDA reaction. Test band intensity was calculated using TOPSE app (Repeated in three independent experiments). FELUDA detection is semi-quantitative (due to stoichiometric binding of FnCas9 RNP: target) and therefore shows a strong negative correlation between Ct values and signal intensities (FIG. 21). This makes it uniquely placed among CRISPRDx platforms to accurately predict the viral load in patient samples with high reproducibility between assays. Through extensive optimization of PCR and reaction components, FELUDA reached a limit of detection (LOD) of ~10 copies of purified viral sequence (FIG. 22, 26). Upon gradual dilution of patient RNA, both FELUDA and qRT-PCR were able to detect samples till the same dilution range (FIG. 23).

### Example 9

### COVID-19 Detection Using 2 Gene FELUDA Assay

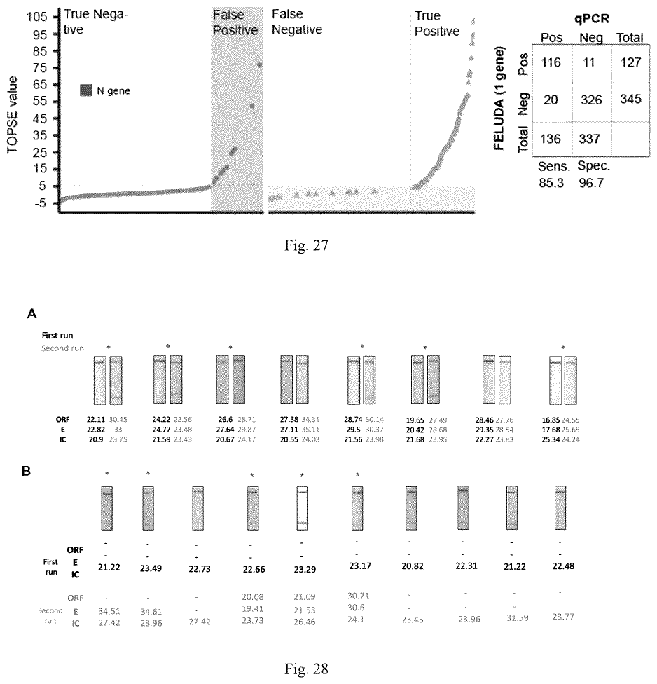

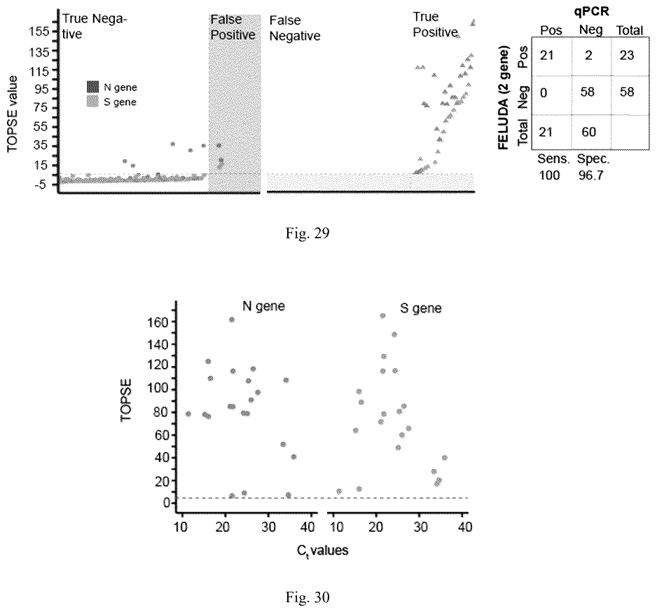

FELUDA using one gene (N), single pass assay was first performed on qRT-PCR confirmed 473 samples and obtained a sensitivity of 85.3% (116/136) and specificity of 96.7% (326/337) with qRT-PCR (FIG. 27). Among the samples that showed discordance between FELUDA with qRT-PCR, 8 false negative FELUDA samples and 10 false positive FELUDA samples were picked up for a repeat evaluation by FELUDA and qRT-PCR. Surprisingly, a repetition of FELUDA with double the amount of RNA yielded positive signals in 6/8 (75%) of samples and a repeat qPCR yielded positive signals in 5/10 (50%) of initially classified negative samples (FIG. 28). This underscores the error rates seen in a single run assay and has been reported elsewhere Bhoyar et al. [BioRxiv doi.org/10.1101/2020.08.10.242677 (2020)]. To improve FELUDA accuracy, assays for both N and S genes were combined, the starting RNA amount doubled and FELUDA was performed with 81 qRT-PCR confirmed samples. A sensitivity of 100% and a specificity of 97% was obtained and it accurately detected samples up to a high Ct value of 37 showing the robustness of the assay (FIG. 29, FIG. 30).

### Example 10

### Home Testing Assay Based on FELUDA

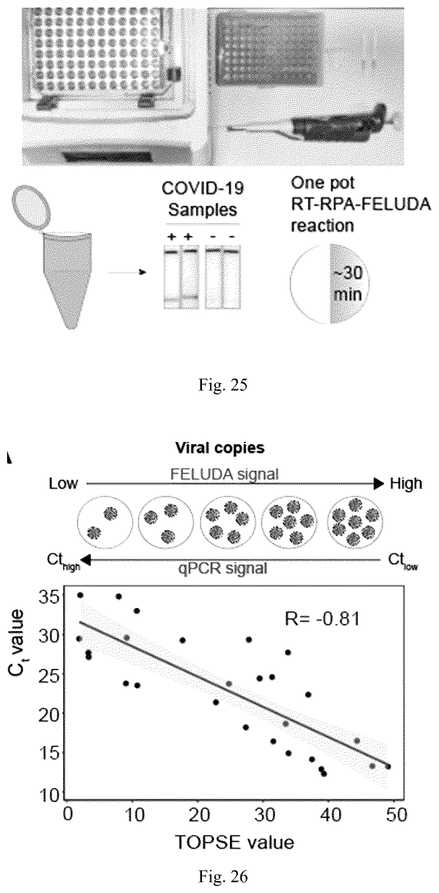

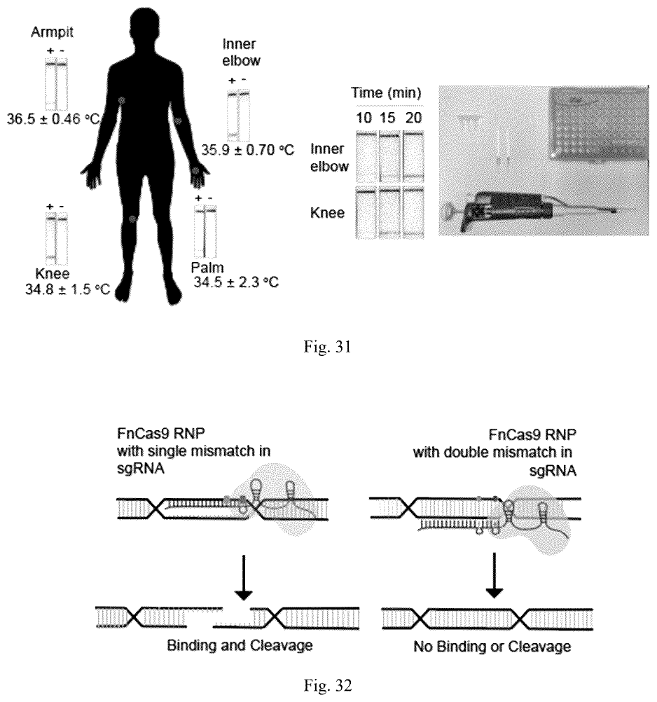

FELUDA can be adapted to a point-of-care or home testing assay Understanding the need for more testing, especially in the wake of rising numbers and predictions of a second wave of infection [Xu et al., Lancet. 395, 1321-1322 (2020)], a PCR machine free version of FELUDA was implemented using Recombinase Polymerase Amplification (RPA) [Zhao et al., Chem. Rev. 115, 12491-12545 (2015); Lobato et al., Trends Analyt. Chem. 98, 19-35 (2018)] that can detect SARS-CoV2 RNA in biological samples within 30 min (FIG. 25 SEQ ID NOs 64-69). Finally, to adapt FELUDA for possible home testing in the future, an on-body 30 min RPA-FELUDA (tested using synthetic RNA fragments) was developed, thus generating an end-to-end instrumentation free testing protocol (FIG. 31).

### Example 11

### FELUDA can be Adapted for any SNV (Development of Bioinformatics Tool JATAYU)

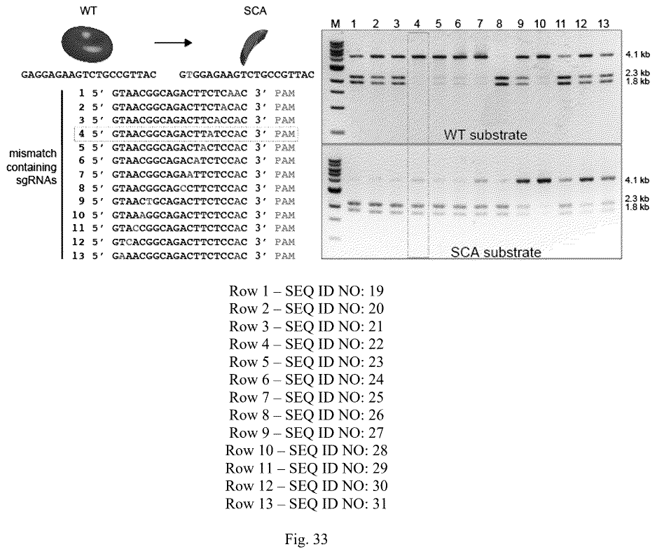

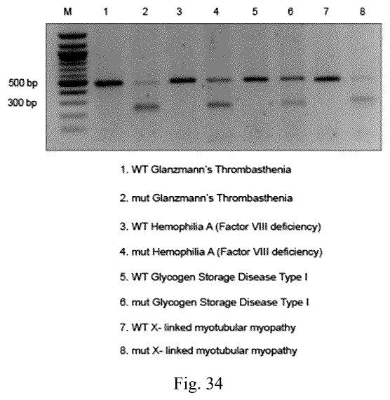

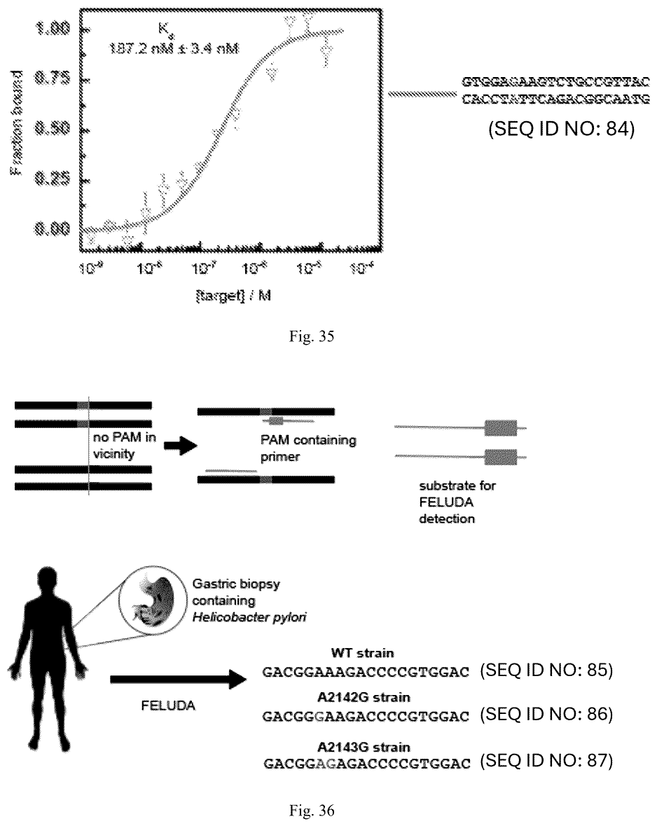

To identify a SNV with high accuracy, it was first investigated if FnCas9 can be directed to cleave the wild type (WT) allele at a SNV by placing an additional MM in the sgRNA sequence specific to the SNV (FIG. 32). To test this, sickle cell anemia (SCA), a global autosomal recessive genetic disorder caused by a point mutation (GAG>GTG) was selected [Soler and Soler Curr. Opin. Hematol. 27 (2020) D. C. Rees, New England J. Med., 1561-1573 (2017)]. By fixing the position of the SNV and walking along the entire length of the sgRNA, it was discovered that two mismatches at the PAM proximal 2nd and 6th positions completely abrogated the cleavage of the SNV target while leaving the WT target intact (FIG. 33, Targets represented by SEQ ID NOs. 19-32 using sgRNAs represented by SEQ ID NOs. 51-63). FELUDA was tested on DNA cloned from 6 SCA patients and a healthy control and obtained a clearly identifiable signature for SNV in every case (FIG. 34 SEQ ID NOs. 36-47). Importantly, the same design principle for searching and discriminating can be universally applied to other Mendelian SNVs without the need for optimization (FIG. 35). To aid users for quick design and implement FELUDA for a target SNV, a web tool JATAYU (Junction for Analysis and Target Design for Your FELUDA assay) has been developed that incorporates the above features and generates primer sequences for amplicon and sgRNA synthesis (https://jatayu.igib.res.in).

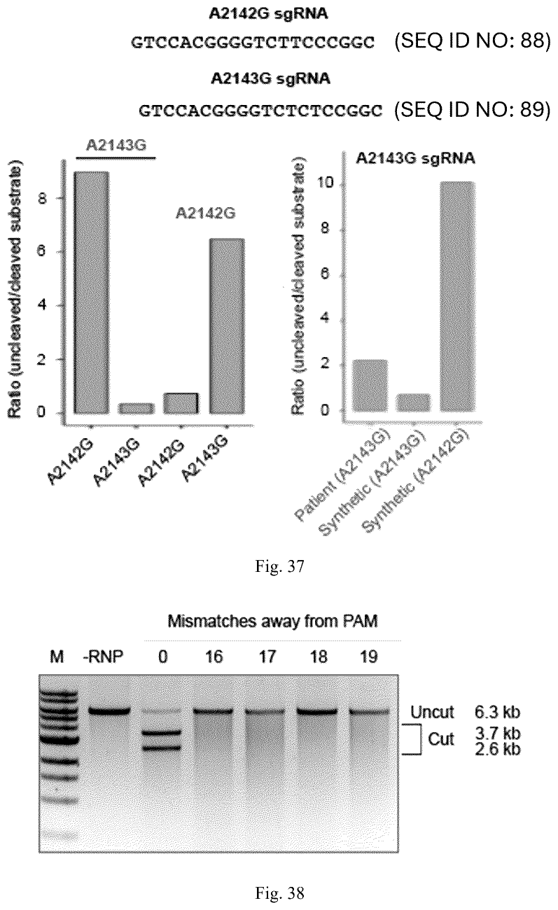

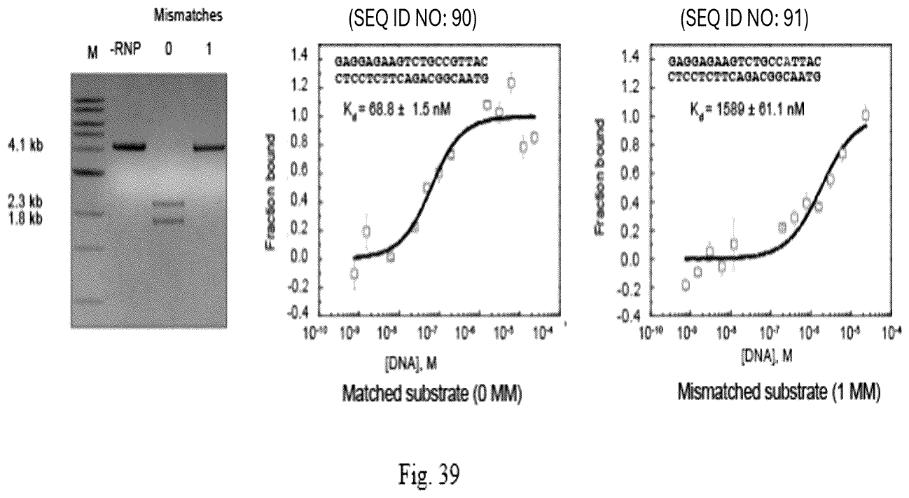

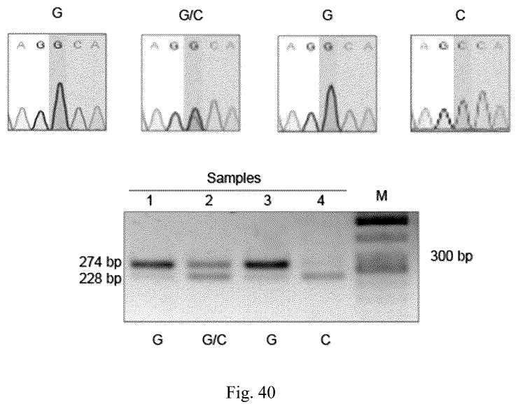

Several improvements were made to FELUDA for expanding and simplifying its detection spectrum. To detect non-PAM proximal SNVs, an in-built PAM site was installed in the amplification step of FELUDA (PAMmer) and successfully validated this approach using 2 SNVs (A2142G and A2143G) present in Helicobacter pylori 23s rRNA gene which confers variable clarithromycin resistance in patients with gastric ulcers [Landrum et al., Nucleic Acids Res. 42, D980-5 (2014); Ribeiro et al., Ann. Clin. Microbiol. Antimicrob. 2, 11 (2003); Binkowska et al., Front. Microbiol. 9, 1-10 (2018)] (FIGS. 36-37) Next, it was tried to reduce PAM dependency on sgRNA design further by exploring if FELUDA could be performed with a single mismatch in the sgRNA. In line with previous reports, it was found that FnCas9 shows negligible cleavage with sgRNAs containing mismatches at the PAM distal end and in particular, mismatch at PAM distal 16th base showed complete absence of cleavage or binding (FIGS. 38-39). This strategy was confirmed by targeting the SNV rs713598 (G>C) in different individuals and successfully identified their genotypes 30 (FIG. 40). It was also shown that FELUDA based detection can work robustly across a wide temperature range and up to 3 days post thawing of reaction components (at room temperature). Thus, field studies using FELUDA can be conducted in diverse climatic conditions and reaction components can be successfully used following cold chain transportation (FIG. 41)

### COMPARATIVE EXAMPLE

To show the higher efficiency of the FnCas9 ribonucleoprotein complex of the present invention comprising a cas9 protein from Francisella novicida and a single single guide RNA (FnCas9:sgRNA) over the known ribonucleoprotein complex in the art (Sp:Cas9 sgRNA), comparative tests were done.

The nucleotide sequence corresponding to the sgRNA for a representative locus for targeting by SpCas9 and its derivatives from Ran et al, (Nat Protoc. 8, 2281-2308 (2013)

(SEQ ID NO: 74)
5′
NNNNNNNNNNNNNNNNNNNNAGGGTTTTAGAGCTAGAAATAGCAAGTTAA
AATAAGGCTAGTCCGTTATCAACTTGAAAAAGTGGCACCGAGTCGGTGCTT
TTTTT 3′

where N stretch corresponds to 20 nucleotides of target sequence of the form

(SEQ ID NO: 75)
GTAACGGCAGACTTCTCCTCAGGGTTTTAGAGCTAGAAATAGCAAGTTAAA
ATAAGGCTAGTCCGTTATCAACTTGAAAAAGTGGCACCGAGTCGGTGCTTT
TTTT

The sgRNA sequence for Hemoglobin locus for FnCas9 from Hirano et al. [Cell, 164(5), 950-961 (2016)]:

(SEQ ID NO: 76)
5′
NNNNNNNNNNNNNNNNNNNNAGGGTTTCAGTTGCGCCGAAAGGCGCTCTG
TAATCATTTAAAAGTATTTTGAACGGACCTCTGTTTGACACGTCTG 3′

where N stretch corresponds to 20 nucleotides of target sequence, of the form

(SEQ ID NO: 77)
5′
GTAACGGCAGACTTCTCCTCAGGGTTTCAGTTGCGCCGAAAGGCGCTCTGT
AATCATTTAAAAGTATTTTGAACGGACCTCTGTTTGACACGTCTG 3′

These sgRNAs show complete binding to the target. In order to efficiently differentiate between 2 targets that are different by 1 mismatch, in the present application, sgRNA was designed in two strategies for FnCas9. The comparative test show that the same strategy of design cannot discriminate between substrates using SpCas9 used in the prior art.

### The First Strategy

The first strategy of mismatch discrimination comprises of 2 mismatches in the FnCas9 sgRNA, at PAM proximal 2nd and 6th position. This was achieved by systematically changing the position of mismatches in the sgRNA in duplex fashion till a completely opposite cleavage outcome was achieved as disclosed in FIGS. 32 and 33. FIG. 32 shows a schematic showing the differences in outcome when FnCas9 encounters a target with 1 mismatch vs 2 mismatch. FIG. 33 shows the positions with 2 mismatches tested on 2 substrates (corresponding to Wildtype, WT Hemoglobin sequence and SCA, Sickle Cell Anemia Hemoglobin sequence). Mismatch positions with respect to PAM (green) are shown in red and the red dotted box shows the most optimal region for placing mismatches that are able to distinguish the two substrates.

The representative sequence of sgRNA for disease detection as provided in the present application (2 mismatches): Mismatches in 2nd and 6th position PAM (AGG) proximal

(SEQ ID NO: 54)
5′
GTAACGGCAGACTTATCCACAGGGTTTCAGTTGCGCCGAAAGGCGCTCTGT
AATCATTTAAAAGTATTTTGAACGGACCTCTGTTTGACACGTCTG 3′

To compare between the efficiency of SNP dissection between this design strategy of the present invention and that of known Cas9 RNP (SpCas9) or its high fidelity derivative (SpCas9-HF1), corresponding sgRNAs with mismatches at identical positions were designed for SpCas9 and SpCas9-HF.

Representative sequence of sgRNA for SpCas9 and SpCas9-HF1 using identical strategy (2 mismatches): Mismatches in 2nd and 6th position PAM (AGG) proximal

(SEQ ID NO: 78)
5′
GTAACGGCAGACTTATCCACAGGGTTTTAGAGCTAGAAATAGCAAGTTAAA
ATAAGGCTAGTCCGTTATCAACTTGAAAAAGTGGCACCGAGTCGGTGC
3′

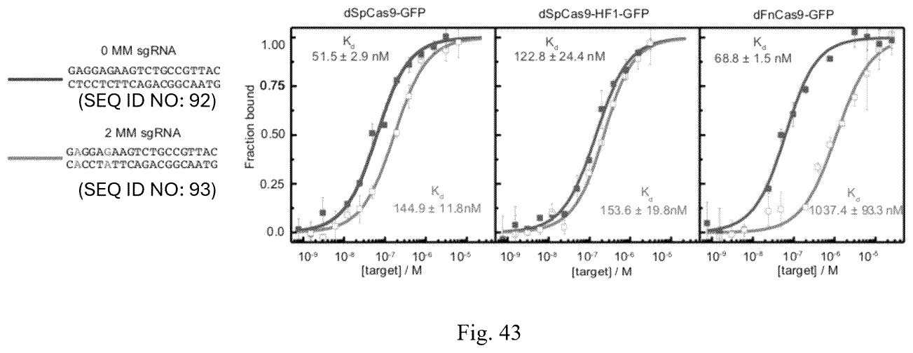

FIG. 43 shows the binding affinity experiments using Microscale Thermophoresis showing interaction of dSpCas9-GFP, dSpCas9-HF1-GFP and dFnCas9-GFP with substrates with 0 (▪) or 2 (∘) mismatch (MM). Values are expressed as fraction bound protein (y-axis) with respect to varying concentrations of purified DNA substrate (Molar units, M, x-axis). Error bars represent SEM (2 independent experiments). It was observed that only in the case of FnCas9, the binding with substrate was lost (very high Kd of 1037.4+/−93.3 nM signifies negligible binding). In case of both SpCas9 and SpCas9-HF1 the kd of mismatched targets are 144.9+/−11.8 nM and 153.6+/−19.8 nM respectively showing very strong binding. All proteins are tagged with GFP to enable fluorescence measurement of binding affinities. These results indicate that the sgRNA design parameters for distinguishing mismatches are specific to FnCas9 and cannot be demonstrated by other proteins reported in prior art.

Taken together, these experiments show that the design principle of the present invention (2 mismatches at PAM proximal 2nd and 6th positions) applies only to FnCas9 and its sgRNA and not for SpCas9 or even its high fidelity derivatives mentioned in literature [Kleinstiver et al. Nature 529, 490-495(2016)].

### The Second Strategy

The second strategy of mismatch discrimination comprises of a single nucleotide position mismatch at the PAM distal end which can also lead to efficient discrimination between 2 substrates as shown below.

FIG. 38 shows cleavage outcomes on a DNA substrate with 1 bp mismatch at PAM distal 16, 17, 18 and 19 positions (sequences provided below). It can be observed that a nucleotide mismatch at each of these positions can render the FnCas9: RNP complex incapable of cleaving a target. Thus a mismatch at any of these positions can be used to distinguish between 2 substrates different by 1 nucleotide. FIG. 39 shows cleavage outcomes on a DNA substrate with 1 bp mismatch at PAM distal 16th position. As it can be observed, a single nucleotide mismatch at the 16th position is able to render the FnCas9:RNP complex incapable of cleaving the same target. Further, the binding affinity experiments using Microscale Thermophoresis show high binding affinity with no mismatches to the substrate and negligible binding affinity when one mismatch at 16th position (PAM distal) is present (MM, mismatches). Values are expressed as fraction bound protein (y-axis) with respect to varying concentrations of purified DNA substrate (Molar units, M, x-axis). Error bars represent SEM (2 independent experiments). Using FnCas9 and a single mismatch (16th position, PAM distal) in the sgRNA, the binding with substrate was lost (very high Kd of 1589+/−61.1 nM signifies negligible binding). The zero (0) mismatch values are shown in the figure.

Taken together, these experiments show that the design principle of the present application i.e. 1 mismatches at PAM distal 16th position can be used to distinguish two substrates different by 1 nucleotide.

Generic FnCas9 sgRNA for disease detection mentioned in present application (1 mismatch): position 16 bp away from PAM denoted by X

(SEQ ID NO: 80)
5′
NNNNXNNNNNNNNNNNNNNNAGGGTTTCAGTTGCGCCGAAAGGCGCTCTG
TAATCATTTAAAAGTATTTTGAACGGACCTCTGTTTGACACGTCTG′
3′

Generic FnCas9 sgRNA for disease detection mentioned in present application (1 mismatch): position 17 bp away from PAM denoted by X

(SEQ ID NO: 80)
5′
NNNXNNNNNNNNNNNNNNNNAGGGTTTCAGTTGCGCCGAAAGGCGCTCTG
TAATCATTTAAAAGTATTTTGAACGGACCTCTGTTTGACACGTCTG′
3′

Generic FnCas9 sgRNA for disease detection mentioned in present application (1 mismatch): position 18 bp away from PAM denoted by X

(SEQ ID NO: 80)
5′
NNNXNNNNNNNNNNNNNNNNAGGGTTTCAGTTGCGCCGAAAGGCGCTCTG
TAATCATTTAAAAGTATTTTGAACGGACCTCTGTTTGACACGTCTG′
3′

Generic FnCas9 sgRNA for disease detection mentioned in present application (1 mismatch): position 19 bp away from PAM denoted by X

(SEQ ID NO: 80)
5′
NNNXNNNNNNNNNNNNNNNNAGGGTTTCAGTTGCGCCGAAAGGCGCTCTG
TAATCATTTAAAAGTATTTTGAACGGACCTCTGTTTGACACGTCTG′
3′

In order to make the discrimination amenable to point of care diagnostic kits such as fluorescence readout or paper strip readout as described in the present invention, the sgRNA was further modified to enable binding based discrimination either by fluorescence or on a paper strip. FIG. 16 shows that adaptation of chimeric gRNA for FELUDA. Cleavage outcomes with different lengths of overlap between crRNA and tracrRNA are shown. 19nt overlap performs most optimally when used in an in vitro cleavage reaction.

To enable fluorescent tagging of the sgRNA, a chimeric sgRNA in two parts with 19 nucleotide overlap is designed (SEQ ID NO: 36 and SEQ ID NO: 37 to be annealed to make full length sgRNA). This chimeric design performed catalytic function similar to that of a full length sgRNA showing no loss of activity due to the split-sgRNA synthesis and also without any loss of specificity for 1 mismatch discrimination. FIG. 42 sows gel showing cleavage outcomes for SARS CoV2 on-target and 1 mismatch off-target for either the single single guide RNA or the chimeric (dual) single guide RNA using FnCas9 RNP. No loss of activity or specificity is seen. A different locus is shown as control.

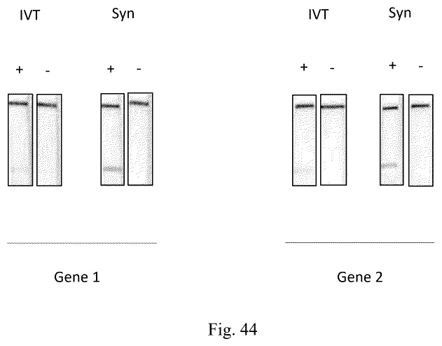

The design was further modified to suit the possibility of detecting polynucleotides on a paper strip using 3′ FAM labelled Part 2 of the gRNA. Additionally, both parts were modified with Phosphorothioate backbone (SEQ ID no. 72, 73). These modifications resulted in more robust, accurate and sensitive detection of SARS CoV2 in patient samples. FIG. 15 shows the outline of lateral flow assay using FELUDA showing positions of control and test bands. FIG. 44 shows the representative images showing differences between unmodified (IVT, in vitro transcribed) vs Phosphorothioate modified (syn, synthetic) gRNA (Genes 1 and 2) [stored at −20 and after undergoing multiple freeze thaw processes].

### Advantages of the Present Invention

The main advantages of the present invention are as follows.
1.) The kit of the present invention comprising the CAS9 CRISPR effector system can be used to identify any mutation in an amplified polynucleotide and produce a read out that can be used for genotyping the target with respect to that single nucleotide variant without the need for sequencing.
2.) The kit of the present invention can be used for rapid, point of care diagnostic for COVID-19 presence in a biological sample.
3.) The time taken for isothermal amplification and enzymatic cleavage is extremely short, leading to very quick detection. Results can be obtained within a couple of hours which allows the initiation of quick prophylactic measures and community transfer steps.
4.) The kit and method of the present invention can be used for multiple targets where the only variable component of the system is the crRNA harboring mismatches to the target. By careful design, the minimum additional binding sequence for CAS9-RNP to the target (namely, the PAM sequence) can be synthetically added to the primers for target amplification, which expands the detection ability to any SNV.
5.) The kit of the present invention is highly specific towards the results with very few false positive or false negative results obtained.
6.) By combining different sgRNA sequences in a systematic manner, the kit of the present invention allows detection of both heterozygous as well as homozygous single nucleotide variants (SNVs). Thus, both carriers as well as diseased individuals can be genotyped for recessive disorders in addition to genotyping homozygous dominant individuals.
7.) As the reaction components of the kit can be assembled in very small quantities, the cost of running the reaction is reduced.
8.) The kit and method of the present invention requires minimum time for sample preparation and hence, can be adapted to low cost, highly efficient, point of care assays that can benefit patients in remote areas without the access to sequencing platforms.

1-33. (canceled)

34. A method for detection of a target polynucleotide comprising:
(a) isolating a polynucleotide;
(b) providing FnCas9 protein with single guide RNA;
(c) providing primers having sequence selected from the group consisting of SEQ ID NOS. 37, 38, 40, 41, 43, 44, 46, 47, 49, 50, 65, 66, 68, 69;
(d) amplifying the isolated polynucleotide of step (a) using Recombinase Polymerase Amplification or PCR with forward and reverse primers obtained in step (c) to obtain a product;
(e) incubating the product obtained in step (d) with a FnCas9 ribonucleoprotein complex obtained in step (b) in a reaction tube comprising a reaction buffer for a time period in the range of 10 to 20 minutes at a temperature in the range of 35 to 37° C.; and
(f) visualizing the results obtained in step (e) by a method selected from the group consisting of agarose gel electrophoresis, lateral flow detection and Fluorescence detection.

35. The method as claimed in claim 34, wherein the target polynucleotide is aDNA or a RNA.

36. The method as claimed in claim 34, wherein the target polynucleotide is a pathogenic or non-pathogenic DNA or RNA.

37. The method as claimed in claim 34, wherein the target polynucleotide is a SARS-COV2.

38. The method as claimed in claim 34, wherein the target polynucleotide is a single nucleotide variant related to a disease selected from the group consisting of, but not limited to, sickle cell anemia, Glanzmann's Thrombasthenia, Glycogen Storage Disease Type I, Hemophilia A, X-linked myotubular myopathy.

39. The method as claimed in claim 34, wherein optionally Reverse transcription is carried out after step (a) when the polynucleotide is RNA.

40. The method as claimed in claim 34, wherein the FnCas9 protein is isolated from Francisella novicida having SEQ ID NO: 2 and 3.

41. The method as claimed in claim 34, wherein the single guide RNA has two parts having SEQ ID NO. 70 and 71.

42. The method as claimed in claim 34, wherein the single guide RNA has a sequence selected from the group consisting of SEQ ID NOS. 32, 33, 36, 39, 42, 45, 48, 51-64, 67, 76, 77, 79, 80.

43. The method as claimed in claim 34, wherein the method of lateral flow detection comprises,
adding a paper strip and dipstick buffer to the reaction tube after step 14 (c); and
incubating the tube obtained in step (a) for a period in the range of 5 to 10 minutes to observe a visible band on the paper strip,
wherein the presence of the band on the paper strip confirms the presence of target polynucleotide.

44. A system for detecting diseases caused by single nucleotide variants (SNVs), wherein the system consists of:
a. Cas 9 effector protein;
b. Sg RNA;
c. PCR primers for amplification of a target;
d. Reverse transcriptase if the sequence is an RNA sequence and amplification enzymes like a Taq polymerase; and
e. Lateral flow assay strip with its buffer,
wherein the Cas9 protein is isolated from Francisella novicida.

45. The system as claimed in claim 44, wherein the Sg RNA is selected from the group consisting of SEQ ID NOS 5, 6, 8, 9, 11, 12, 14, 15, 17, 18, 19-36, 39, 42, 45, 48, 64, 67, 76, 77, 79, 80.

46. The system as claimed in claim 44, wherein the primers are selected from the group consisting of SEQ ID NOS. 37, 38, 40, 41, 43, 44, 46, 47, 49, 50, 65, 66, 68, 69.

47. The system as claimed in claim 44, wherein the Cas9 protein is fluorescent labeled.

48. The system as claimed in claim 44, wherein the target nucleic acid is selected from DNA and RNA having SEQ ID NOS. 4, 7, 10, 13, 16.

49. Use of the The system as claimed in claim 44, wherein the for detecting diseases are selected from the group comprising of Genetic disorders like sickle cell anemia, Glanzmann's Thrombasthenia, Glycogen Storage Disease Type I, Hemophilia A, X-linked myotubular myopathy and Pathogenic diseases caused by microbes selected from the group comprising of SARS-CoV2, SARS CoV1, Helicobacter pylori infection.

50. The system as claimed in claim 49, wherein the Genetic disorders include sickle cell anemia, Glanzmann's Thrombasthenia, Glycogen Storage Disease Type I, Hemophilia A, and X-linked myotubular.

51. The system as claimed in claim 49, wherein the Pathogenic diseases caused by microbes include SARS-CoV2, SARS CoV1, and Helicobacter pylori infection.
